(19) United States
(12) Patent Application Publication
(10) Pub. No.: US 2021/0295971 A1
(43) Pub. Date: Sep. 23, 2021

(54) SENSITIVE DRUG DISTRIBUTION SYSTEMS AND METHODS

(71) Applicant: Jazz Pharmaceuticals, Inc., Palo Alto, CA (US)

(72) Inventors: Sherice Reneé Mills, San Carlos, CA (US); Jerry Yu, San Diego, CA (US)

(21) Appl. No.: 16/826,909

(22) Filed: Mar. 23, 2020

(51) Int. Cl. G16H 20/10; G16H 15/00; G16H 40/67; G06Q 30/00; G06Q 50/26; G06N 20/00

(52) U.S. Cl. CPC G16H 20/10; G16H 15/00; G06N 20/00; G06Q 30/018; G06Q 50/265; G16H 40/67

(57) ABSTRACT

A computerized system distributes a plurality of different prescription drugs under exclusive control of an exclusive central pharmacy. The central pharmacy includes an exclusive computer database, which in addition to having information relating to all prescription requests, contains information about all doctors permitted to prescribe the different prescription drugs and all patients permitted to receive the different prescription drugs. The system checks the exclusive computer database for potential abuse of any specific prescription drug by a patient or a medical doctor prescribing the specific prescription drug, and sends the specific prescription drug to the patient only if no potential abuse is found by both the patient and the medical doctor.

### RELATED APPLICATIONS

This application is related to U.S. application Ser. No. 16/032,501, filed on Jul. 11, 2018, which claimed priority to U.S. Provisional Application No. 62/534,892, filed on Jul. 20, 2017, and also claimed priority to U.S. Provisional Application No. 62/607,221, filed on Dec. 18, 2017, all of which are related to U.S. application Ser. No. 14/196,603, filed on Mar. 4, 2014, which is a Continuation of and claimed priority to U.S. application Ser. No. 13/592,202, filed on Aug. 22, 2012 and issued on May 20, 2014 as U.S. Pat. No. 8,731,963, which is a Continuation of and claimed priority to U.S. application Ser. No. 13/013,680, filed on Jan. 25, 2011, which is a Continuation of and claimed priority to U.S. application Ser. No. 12/704,097, filed on Feb. 11, 2010 and issued on Feb. 22, 2011 as U.S. Pat. No. 7,895,059, which is a Continuation of and claimed priority to U.S. application Ser. No. 10/322,348, filed on Dec. 17, 2002 and issued on Feb. 23, 2010 as U.S. Pat. No. 7,668,730, which applications and patents are herein incorporated by reference in their entireties.

### TECHNICAL FIELD

The technical field of this disclosure generally relates to the distribution of drugs, and in particular, to the distribution of a plurality of different sensitive drugs. Additionally, an embodiment relates to using an exclusive database to monitor and control the distribution of the plurality of sensitive drugs.

### BACKGROUND

Sensitive drugs are controlled to minimize risk and ensure that they are not abused, or cause adverse reactions. Such sensitive drugs are approved for specific uses by the Food and Drug Administration, and must he prescribed by a licensed physician in order to be purchased by consumers. Some drugs, such as cocaine and other common street drugs, are the object of abuse and illegal schemes to distribute for profit. Some schemes include doctor shopping, diversion, and pharmacy thefts. A locked cabinet or safe is a requirement for distribution of some drugs.

Certain agents, such as gamma hydroxy butyrate (GHB), are also abused, yet also are effective for therapeutic purposes such as treatment of daytime cataplexy in patients with narcolepsy. Some patients however, will obtain prescriptions from multiple doctors, and have them filled at different pharmacies. Still further, an unscrupulous physician may actually write multiple prescriptions for a patient, or multiple patients, who use cash to pay for the drugs. These patients will then sell the drug to dealers or others for profit. Such certain agents can be in different formulations of a particular drug, and/or in a different drug altogether, which can increase the chances of abuse, misuse, and/or diversion of the drug.

There is a need for a distribution system and method that directly addresses these abuses, especially when multiple formulations of a drug exist. There is a further need for such a system and method that provides education and limits the potential for such abuses for multiple formulations and multiple different drugs.

### SUMMARY

A drug distribution system and method utilize a central pharmacy and database to track all prescriptions for a plurality of different sensitive drugs. This plurality can include different formulations of a drug that all share the same active ingredient, or completely different drugs that have different active ingredients. The drugs may be owned by one or more parties and have separate and different reporting obligations. :Information is kept in a central database regarding all physicians allowed to prescribe the sensitive drugs and all patients receiving the drugs. In an embodiment, a caregiver is associated with the patient, such as when the patient is a pediatric patient. Abuses are identified by monitoring data in the database for prescription patterns by physicians and prescriptions obtained by patients. Further verification is made that the physician is eligible to prescribe the drug by consulting a separate database for a valid DEA license, and optionally state medical boards to determine whether any corrective or approved disciplinary actions relating to controlled substances have been brought against the physician. Multiple controls beyond those for traditional drugs are imposed on the distribution depending on the sensitivity of the drug.

Education is provided to the physician and patient. Prior to shipping the drug for the first time, the patient is contacted to ensure that product-related and abuse-related educational materials have been received and/or read. The patient may provide the name of a designee to a central pharmacy who is authorized to accept shipment of the drug. Receipt of the initial drug shipment is confirmed by contacting the patient. Either a phone call or other communication to the patient within a set time after delivery may be made to ensure receipt. Further, a courier service's tracking system is used to confirm delivery in additional embodiments. If a shipment is lost, an investigation is launched to find it.

Prescription refills are permitted in the number specified in the original prescription. In addition, if a prescription refill is requested by the patient prior to the anticipated due date, such refills will be questioned. A lost, stolen, destroyed or spilled prescription/supply is documented and replaced to the extent necessary to honor the prescription, and will also cause a review or full investigation.

An exclusive central database that is associated with the central pharmacy contains all relevant data related to distribution of the drug and the process of distributing it, including patient, caregiver (if one is involved), physician, and prescription information. Several queries and reports are run against the database to provide information which might reveal potential abuse of the sensitive drug, such as early refills.

### BRIEF DESCRIPTION OF THE DRAWINGS

FIG. 1 is a block diagram of a computer system for use in implementing a sensitive drug distribution system.

FIG. 2 is a flowchart describing a method for sensitive drug distribution at least partially utilizing a computer system such as that shown in FIG. 1.

FIG. 3 is a flowchart describing another method for sensitive drug distribution at least partially utilizing a computer system such as that shown in FIG. 1.

FIG. 4 is a flowchart describing another method for sensitive drug distribution at least partially utilizing a computer system such as that shown in FIG. 1.

FIGS. 5A-5C are a flowchart describing another method for sensitive drug distribution at least partially utilizing a computer system such as that shown in FIG. 1.

FIGS. 6A-6B are a flowchart describing another method for sensitive drug distribution at least partially utilizing a computer system such as that shown in FIG. 1.

FIG. 7 is a flowchart describing another method for sensitive drug distribution at least partially utilizing a computer system such as that shown in FIG. 1.

FIG. 8 is a block diagram of database fields.

FIG. 9 is a block diagram showing a list of queries against the database fields.

FIGS. 10A-10F are an example of a patient counseling checklist.

FIG. 11 is an example of a prescription and enrollment form.

FIG. 12 is a copy of certificate of medical need.

FIGS. 13A-13C are descriptions of sample reports obtained by querying a central database having fields represented in FIG. 8.

### DETAILED DESCRIPTION

In the following description, reference is made to the accompanying drawings that form a part hereof and in which is shown by way of illustration specific embodiments in which the invention may be practiced. These embodiments are described in sufficient detail to enable those of ordinary skill in the art to practice the invention, and it is to be understood that other embodiments may be utilized and that structural, logical and electrical changes may be made without departing from the scope of the present invention. The following description is, therefore, not to be taken in a limited sense, and the scope of the present invention is defined by the appended claims.

The following patents, publications and application are related to the present disclosure and are hereby incorporated by reference in their entireties for all purposes: U.S. Pat. Nos. 6,472,431; 6,780,889; 7,262,219; 8,263,650; 8,461,203; 8,859,619; 9,539,330; 7,851,506; 8,324,275; 8,952,062; 8,731,963; 8,772,306; 8,952,029; 9,050,302; 9,486,426; 10,213,400; 8,591,922; 8,901,173; 9,132,107; 9,555,017; 10,195,168; 8,778,301; 9,801,852; 8,771,735; 8,778,398; 9,795,567; and U.S. Patent Publication No. US 2018/0042855 and Application No. 62/953,288.

The functions or algorithms described herein are implemented in software and hardware. The software comprises computer executable instructions stored on computer readable media such as memory or other types of storage devices. The term “computer readable media” is also used to represent carrier waves on which the software is transmitted. Further, such functions correspond to modules, which are software, hardware, firmware of any combination thereof. Multiple functions are performed in one or more modules as desired, and the embodiments described are merely examples. The software is executed on a digital signal processor, ASIC, microprocessor, or other type of processor operating on a computer system, such as a personal computer, server or other computer system.

A sensitive drug is one which can be abused, or has addiction properties or other properties that render the drug sensitive. One example of such a drug is sodium oxybate, also known as gamma hydroxy butyrate (GHB C₄H₇NaO₃), which is useful for treatment of cataplexy in patients with narcolepsy. GHB is marketed under the trademark of Xyrem® (sodium oxybate oral solution), which trademark can be used interchangeably with GHB herein. Xyrem® can exist in more than one formulation. For example, Xyrem® can be embodied in a standard formulation, and it can be embodied in a low sodium formulation. Sensitive drugs also include narcotics or other drugs that require controls on their distribution and use to monitor behaviors to prevent abuse and adverse side effects.

In one embodiment, the different formulations of Xyrem® are subject to a restricted distribution program. One aspect of the program is to educate physicians and patients about the risks and benefits of Xyrem®, including support via ongoing contact with patients and a toll-free helpline. Another aspect of the program is to counsel patients on the different considerations of the administration of the different formulations of Xyrem® or other formulations with the same active ingredient, GHB. Those other formulations include different GHB salts, such as potassium, calcium, magnesium and so on. Salt forms of GHB are disclosed in U.S. Pat. Nos. 8,591,922; 8,901,173; 9,132,107; 9,555,017; and 10,195,168, which are hereby incorporated by reference in their entireties for all purposes. Initial prescriptions are filled only after a prescriber and patient have received and read the educational materials. Further, patient, caregiver (if involved, who is associated with a pediatric, elderly, or other special needs patient), and prescribing physician registries are maintained and monitored to ensure proper distribution.

In a further embodiment, bulk sodium oxybate is manufactured at a single site, as is the different formulations of the finished drug product. Following manufacture of the drug product, it is stored at a facility compliant with FDA Schedule III regulations, where a consignment inventory is maintained. The inventory is owned by a company, and is managed by a central pharmacy, which uses an exclusive central computer database and which maintains the consignment inventory. Xyrem® is distributed and dispensed through a primary and exclusive central pharmacy, which as noted uses an exclusive central database, and is not stocked in retail pharmacy outlets. It is distributed by overnight carriers, or by U.S. mail in one embodiment to potentially invoke mail fraud laws if attempts of abuse occur.

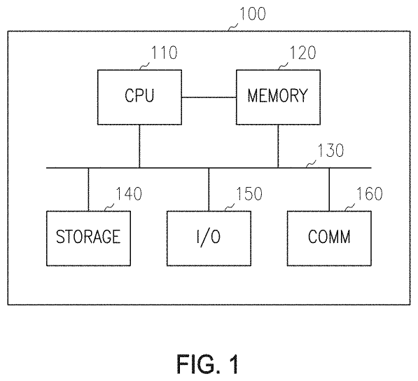

FIG. 1 is a simplified block diagram of a computer system 100, such as a personal computer for implementing at least a portion of the methods described herein. A central processing unit (CPU) 110 executes computer programs stored on a memory 120. Memory 120 in one embodiment comprises one or more levels of cache as desired to speed execution of the program and access to data on which the programs operate. The CPU is directly coupled to memory 120 in one embodiment. Both CPU 110 and memory 120 are coupled to a bus 130. A storage 140, I/O 150 and communications 160 are also coupled to the bus 130. Storage 140 is usually a long term storage device, such as a disk drive, tape drive, DVD, CD or other type of storage device. In one embodiment, storage 140 is used to house a database for use with one or more embodiments of the present invention. I/O 150 comprises keyboards, sound devices, displays and other mechanisms by which a user interacts with the computer system 100. Communications 160 comprise a network, phone connection, local area network, wide area network or other mechanisms for communicating with external devices. Such external devices comprise servers, other peer computers and other devices. In one embodiment, such external devices comprise a database server that is used in place of the database on storage 140. Other computer system architectures capable of executing software and interacting with a database and users may also be used. Appropriate security measures such as encryption are used to ensure confidentiality. Further, data integrity and backup measures are also used to prevent data loss.

In many instances, sensitive drugs, such as Xyrem® and its multiple formulations (and other formulations that have the same active ingredient, GHB), must be distributed under an umbrella of a Risk Evaluation and Mitigation Strategy (REMS) program. A purpose of a REMS program is to mitigate the risks of serious adverse outcomes resulting from inappropriate prescribing, misuse, abuse, and/or diversion of the sensitive prescription drugs, such as Xyrem® and its multiple formulations, which as noted above, are used for the treatment of cataplexy in patients with narcolepsy and for the treatment of excessive daytime sleepiness (EDS) in patients with narcolepsy. This goal is achieved by 1) informing prescribers, pharmacists, patients, and caregivers (if one is involved) of the risks, contraindications, potential for abuse, misuse, and overdose, and the safe use, handling, and storage of the particular formulation of the sensitive drug, and 2) ensuring that central pharmacy controls exist prior to filling prescriptions, wherein the controls screen for concomitant use of sedative hypnotics and other potentially interacting agents, monitor for inappropriate prescribing, and monitor and notify prescribers about concomitant use of contraindicated medications or signs of potential abuse, misuse, and/or diversion. One feature of such a REMS program is a single, certified, central pharmacy, and its associated exclusive computer database, that handles all prescription requests for all different drugs and different formulations, written by all prescribers, for all patients being prescribed the sensitive drugs. To become certified, all pharmacy staff and pharmacists of the central pharmacy must have completed a REMS pharmacy training program. The exclusive central database contains information about patient, caregiver, and prescriber enrollments, prescriptions for sensitive drugs, sensitive drug shipments, and Risk Management Reports (RMRs) generated by the certified, central pharmacy whenever there is activity raising a suspicion of abuse, misuse, and/or diversion, including requests for early refills.

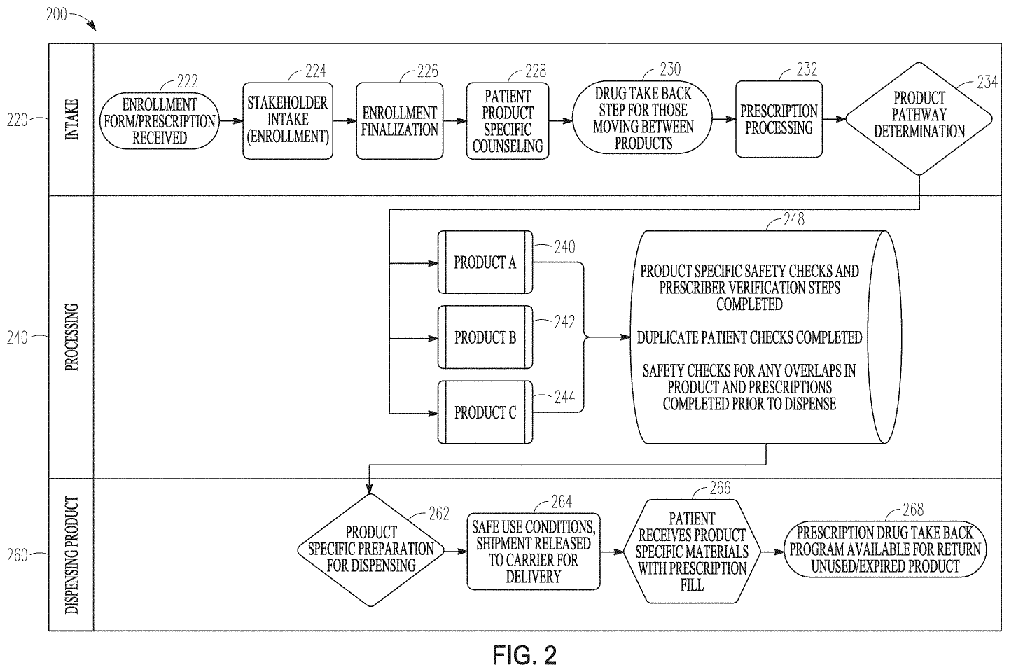

All patients must be enrolled in the REMS program before they can receive a sensitive drug shipment. Additionally, as noted below in connection with FIG. 2, an enrolled prescriber completes an enrollment form for the patient and submits the form to the REMS program for processing and completion of enrollment. This form provides the patient's demographic and insurance information, links an enrolled prescriber to an enrolled patient, and verifies that specified requirements have been met, including an acknowledgment that the prescriber has counseled the patient on the serious risks and safe use of the sensitive drug and the particular formulation of the sensitive drug. This information is entered into the exclusive database of the certified, central pharmacy, thereby allowing the pharmacy to verify that the patient is enrolled as part of its validation process prior to dispensing the sensitive drug. Additionally, all prescriptions for the sensitive drug go directly from an enrolled prescriber to the REMS Program, which limits an individual's ability to forge prescriptions or secure uncontrolled amounts of the sensitive drug with multiple prescriptions from different prescribers. As a further precaution, the sensitive drug prescription form is only available directly from the REMS program, not indirectly from the REMS website or other secondary source. The prescriber is notified once a patient is successfully enrolled and can be prescribed the sensitive drug.

Prescribers are considered to be enrolled in the REMS program only after verification that the prescriber enrollment form has been completed and the requirements described on the form have been met. Enrollment information is entered into the exclusive database of the certified, central pharmacy so that the pharmacy can verify that the prescriber is enrolled in the REMS program as part of its validation process prior to accepting a sensitive drug prescription or dispensing the sensitive drug. The certified, central pharmacy fills prescriptions only from prescribers who have completed enrollment in the REMS program. Prescribers are not enrolled (and therefore their patients cannot receive the sensitive drug) until the prescribers verify that they have reviewed educational materials and understand the approved indications and risks of the sensitive drugs and their different formulations and complete the prescriber enrollment form. Upon receipt of an initial prescription, the certified, central pharmacy checks the exclusive database to ensure that the prescriber is enrolled. If the prescriber is enrolled, the certified, central pharmacy will verify the prescriber's name, DEA number, and state license number using appropriate sources. The DEA and license confirmation ensures that only prescriptions from enrolled prescribers who can legally prescribe a controlled substance are filled.

The certified, central pharmacy tracks each shipment of a formulation of a sensitive drug through a carrier's or delivery service's website to confirm shipment within a particular time frame, such as one business day. Tracking reports are generated to confirm the receipt of orders shipped during the previous 48 hours. If an order is not delivered on the initial attempt, the carrier can make multiple attempts to redeliver. If redelivery attempts are not successful, the carrier returns the shipment to the certified, central pharmacy, per its arrangement with the pharmacy. The returned product is inspected for evidence of damage or tampering, and it is noted in the exclusive database whether the product is acceptable for reshipment (only applicable if the product has not been delivered and has remained in the carrier's possession and reshipment is only to the same patient). In the event a shipment is lost by the carrier, a replacement shipment cannot be sent until approved by a pharmacist-in-charge at the certified, central pharmacy.

The certified, central pharmacy ensures that only one sensitive drug prescription for only one formulation is being dispensed at any one time to any one patient. Because the certified, central pharmacy contacts the prescriber to renew a prescription before the previous prescription has expired (to ensure continuity of treatment for the patient), new prescriptions often arrive before the current prescription has expired. When a new prescription arrives for a patient who already has an active prescription, the new prescription is put on hold until the prescription already in the exclusive database is depleted. If the certified, central pharmacy receives a prescription for a patient from more than one prescriber, certified, central pharmacy staff will know that the patient already has an active prescription in the exclusive database and will query the patient as to the reason for having more than one prescriber write prescriptions for the patient. If the patient has more than one prescription from more than one prescriber, the certified, central pharmacy will contact each prescriber for clarification. Depending on the clarification received from the different prescribers, the certified, central pharmacy will either terminate the current prescription prior to activating the new one or will put the new prescription on hold until the current prescription has been depleted. If there is a reasonable suspicion that a patient may be doctor shopping for purposes of abuse, misuse, and/or diversion, the prescription will not be filled, the prescriber(s) will be alerted, and the pharmacist at the certified, central pharmacy will complete an RMR.

Upon receipt of the initial prescription for every patient, the certified, central pharmacy verifies the patient's enrollment and checks the patient's profile in the exclusive database to ensure the patient has not previously enrolled. For each initial prescription, at the time of data entry, the certified, central pharmacy searches the database by last name, city and state, social security number (if provided), and date of birth to verify patient enrollment status. If there are no matches, the pharmacy will proceed with processing this patient as a new enrollment. In some instances, a patient may be enrolled under more than one name (for example, if the patient's name changes because of marriage). When a duplicate patient profile is detected, the certified, central pharmacy ensures that only one profile is active and eligible to receive shipments.

The certified, central pharmacy does not allow shipment to patients who have not enrolled in or have been removed from the REMS program or have stopped treatment using the sensitive drug. Upon receipt of each initial prescription, the certified, central pharmacy checks the enrollment status of each patient and prescriber. At each refill, the certified, central pharmacy contacts the patient to update the patient profile in the exclusive database, which provides another check on enrollment status.

As noted above, a tool used at the certified, central pharmacy to monitor for inappropriate prescribing, misuse, abuse, and/or diversion of the sensitive drug is a risk management report (RMR). Investigations into potential abuse, misuse, and/or diversion can be made a requirement of the REMS program, and the RMR procedures can be a requirement included in the certified, central pharmacy's elements to assure safe use (ETASU) of the REMS program. A pharmacist at the certified, central pharmacy must complete an RMR for any event that gives rise to a reasonable suspicion of abuse, misuse, and/or diversion of the sensitive drug. This includes all requests for an early refill, reports of potential misuse, abuse, and/or diversion, and all reports of lost, stolen, destroyed, or spilled drug.

A completed RMR includes patient identifier and profile information, the date of the event, a descriptive narrative of the event, a record of the certified, central pharmacy contact(s) with the prescriber related to the event, the outcome(s) of the event including any action taken by the certified, central pharmacy related to the event, and a list of any documents or reports related to the event (e.g., adverse event reports, police reports, DEA reports for lost shipments, and/or fire reports). In many cases, RMRs will document investigations that include multiple pharmacy-patient, pharmacy-caregiver, and/or pharmacy-prescriber interactions that transpire over several days. The completed RMR provides a concise compilation of relevant information associated with a potential risk event, and when documented in the exclusive database, provides longitudinal data that the certified, central pharmacy can consult to determine whether a patient's pattern of behavior indicates a likelihood of abuse, misuse, and/or diversion.

The RMR history for each enrolled patient who is receiving a sensitive drug from the certified, central pharmacy is available for consultation and review at the pharmacy, and this review is required as part of a pharmacist's handling of an early refill request or any time the pharmacist suspects abuse, misuse, and/or diversion of the sensitive drug. In addition, the REMS program requires that the pharmacist at the certified, central pharmacy notifies a prescriber in the event of suspected abuse, misuse, and/or diversion of the drug and when a patient has requested an early refill. Any information contained in a patient's RMR history provides an organized summary of previous patient experiences and problems with the drug that can be reviewed with the prescriber to ensure awareness of individual patient behaviors and the possible need for additional patient counseling or other actions, ranging from restrictions on early refills to removal from the REMS program.

Alerts on a patient's and/or caregiver's profile stored in the exclusive database help to direct any subsequent interaction with the patient and/or alert all pharmacy staff of the certified, central pharmacy that this patient or caregiver has a history of potential abuse, misuse, and/or diversion of the sensitive drug and that requests for early refills or reports of lost or stolen drug or other irregularities should be met with greater scrutiny. In the event that a patient changes his or her prescriber, the certified, central pharmacy provides longitudinal information about the patient, including any relevant alerts, to the new prescriber. In addition, pharmacists at the certified, central pharmacy review prior alerts as part of the RMR procedure, ensuring that both the prescriber and pharmacist are aware of a patient's and/or caregiver's history of possible abuse, misuse, and/or diversion.

An early refill request occurs when a patient, caregiver, and/or prescriber report that the patient will not have sufficient quantities of the sensitive drug to last until the next scheduled shipment, and therefore will require drug to be shipped sooner than scheduled. Early refill requests do not include changes to scheduling that are a result of shipment logistics, such as the carrier not shipping on weekends and holidays. In these cases, the product is delivered prior to a patient's next shipment date, but the patient's subsequent refill date does not shift to ensure the patient does not receive more than is prescribed.

The pharmacist at the certified, central pharmacy is required to complete an RMR for every early refill request, notify the prescriber of the request, and monitor a patient's and/or caregiver's history of early refill requests as part of the required monitoring of patients and/or caregivers for abuse, misuse, and/or diversion of the sensitive drug.

Under the REMS program, prescribers are notified regarding concomitant use of contraindicated medications and serious changes in the patient's medical condition. Notifications are documented in the certified, central pharmacy exclusive database. As noted, the REMS program also documents the number of notifications to prescribers for suspected abuse, misuse, and/or diversion, for alerts regarding potential abuse, misuse, and/or diversion on the patient's profile; for early refill requests; and for prescription errors in a way that is accessible for reporting.

Pharmacists at the certified, central pharmacy are required to notify the prescriber of every early refill request. As early refill requests, including repeated requests, may be a sign of abuse, misuse, and/or diversion, pharmacists are required to review the patient's and/or caregiver's RMR history and any alerts, and ensure that the request has been discussed with the prescriber prior to approving the early refill.

To ensure that the informational objectives of the REMS program are achieved, the REMS program requires all prescribers and the certified, central pharmacy to become specially certified in the REMS program, and further requires all patients and caregivers to enroll in the REMS program. Briefly, prescriber certification requires each prescriber to attest that that he or she has read the prescribing information and a REMS program prescriber brochure, and pharmacy certification requires that the pharmacy must, among other things, ensure that all pharmacists complete a REMS program certified pharmacy training program. The pharmacy training program ensures that all pharmacy staff are trained on the risks, safe use, handling, and storage of the sensitive drug and on the requirements of the REMS program, including completing knowledge assessments. The review of these educational and training materials ensures that all prescribers and pharmacists are informed of the risks and safe use of the sensitive drug. Patient and caregiver enrollment (or an association of the caregiver with the patient) requires that patients and caregivers be informed, during the enrollment process by their prescriber, of the risks of the sensitive drug. This informing can be by way of the information contained in a REMS program patient quick start guide, a medication guide, a caregiver brochure that is specific to pediatric patients (or elderly or other special needs patients), and through completion of a REMS program counseling checklist.

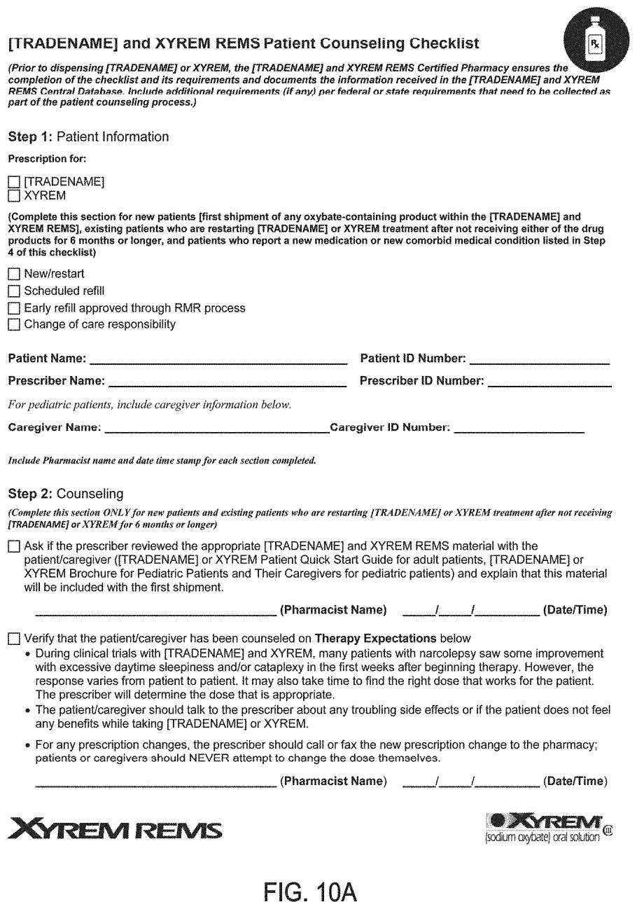

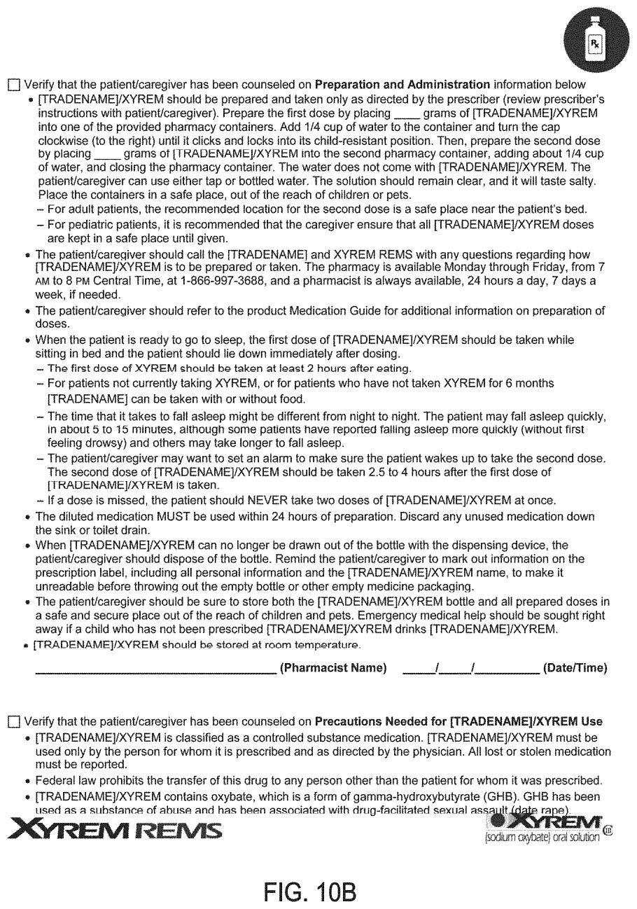

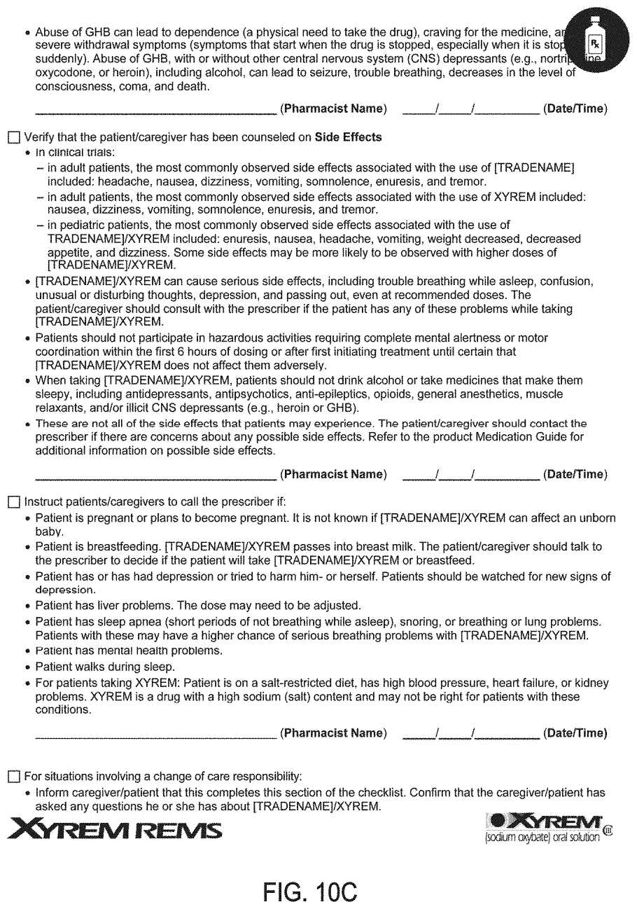

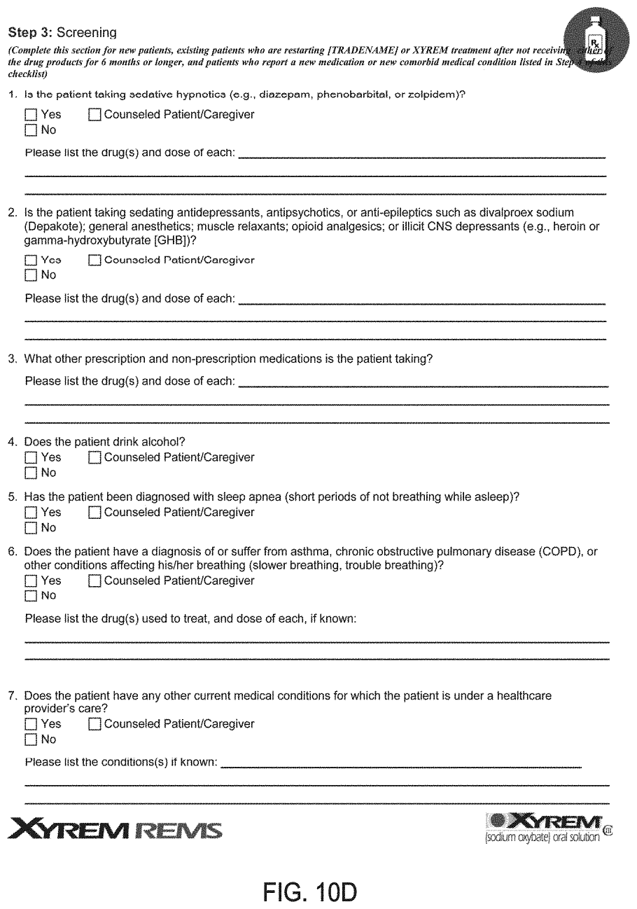

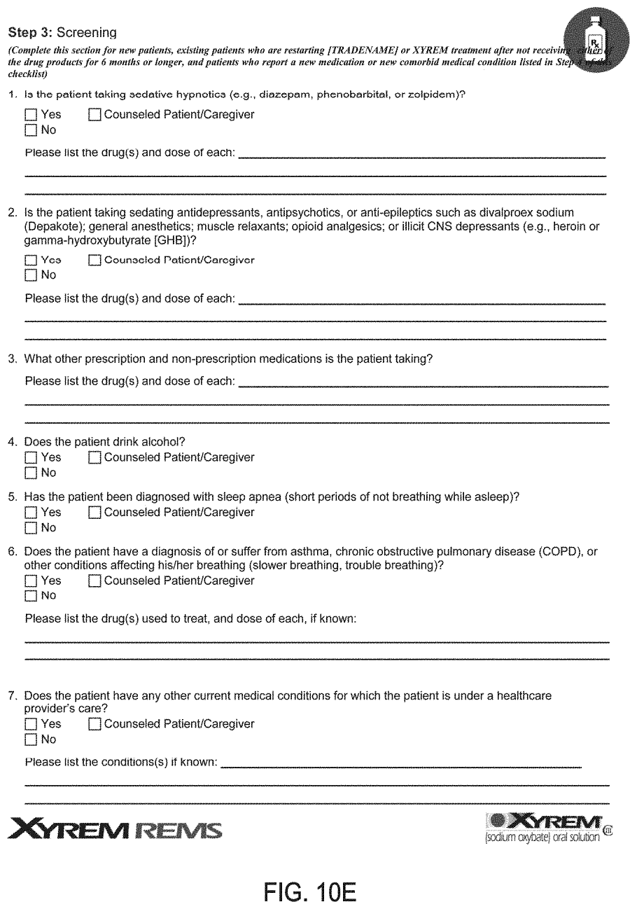

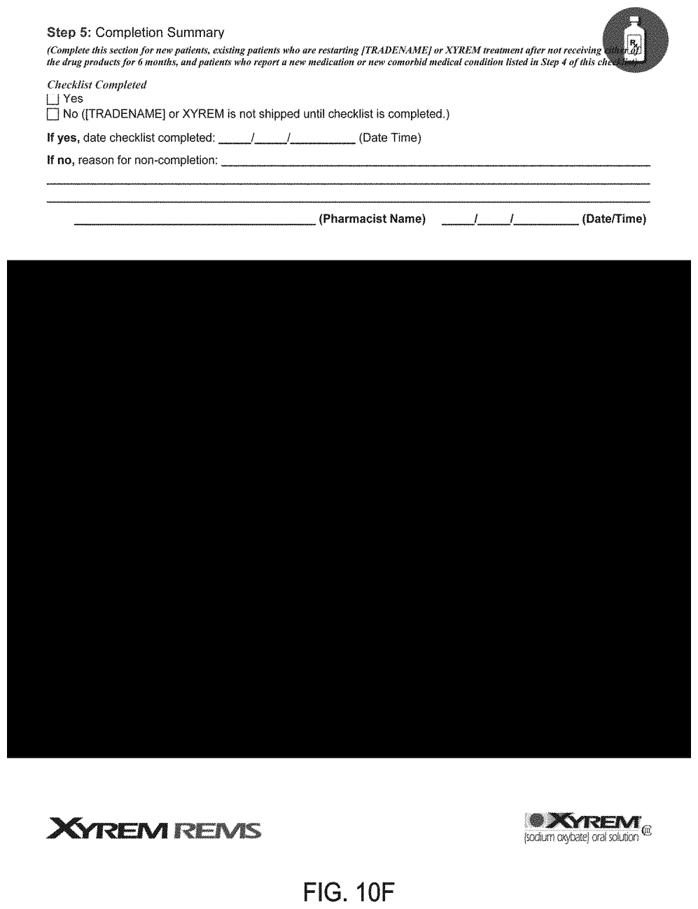

Regarding the counseling checklist, prior to dispensing the sensitive drug, a pharmacist at the certified, central pharmacy contacts the patient and completes the REMS program patient counseling checklist, and ensures that the information received is maintained in the REMS program exclusive database of the certified, central pharmacy. The REMS program patient counseling checklist is the primary tool used by the certified, central pharmacy to counsel newly enrolled patients before their first shipment of the sensitive drug and patients restarting use of the sensitive drug after not receiving the sensitive drug for six months or more. The pharmacist will counsel the patient on the checklist. An example of a counseling checklist is illustrated in FIG. 10.

In addition to the checklist, the exclusive database includes and maintains patient and prescriber enrollment status, all completed REMS forms, prescription and shipment data, as well as information related to dosing, concomitant medications, and behavior that raises suspicion of misuse, abuse, and/or diversion, including complete RMR histories and alerts regarding potential abuse, misuse, and/or diversion on the patient profiles. The exclusive database also contains database-enabled controls and checks to ensure compliance with the REMS program.

More specifically, the information contained in the exclusive database includes the following:

Prescription date, titration and dosing, and prescriber instructions.

Prescriber information, including all current patients who are prescribed a sensitive drug and whether the prescriber's DEA and state license numbers are active.

Patient information, including name and two identifiers, current and previous prescribers, comorbid conditions, concomitant medications and other potentially interacting agents, prescription history, and early refill requests and outcomes.

Certified, central pharmacy interactions with prescribers, patients, caregivers, and other parties regarding the patient, caregiver, prescriber, or prescription, including information that might suggest potential patient, caregiver, and/or prescriber misuse, abuse, and/or diversion.

Data and information on all shipments of the sensitive drug, including number and quantity of shipments sent daily.

Prior to dispensing a newly enrolled patient's first-time fill, a pharmacist at the certified, central pharmacy completes the patient counseling checklist, asking each patient for a list of his or her current medications and doses. The certified, central pharmacy also asks a patient if his or her medications have changed at the time of the scheduling call for subsequent shipments, and a pharmacist must notify the prescriber when the patient is receiving concomitant contraindicated medications.

Upon implementation of the REMS program, patient-reported changes in concomitant medications during any refills and notifications to prescribers are captured through the completion of the REMS program patient counseling checklist. Capture of these medications, with the subsequent confirmation regarding prescriber notification in the checklist, enables accurate reporting of data from both first fills and refills for assessment purposes.

FIG. 2 is a diagram illustrating a system and process to safely distribute to patients a plurality of different types of prescription drugs that are susceptible to abuse, misuse, and/or diversion. As noted above, such different prescription drugs can have the same active ingredient, but they can be different in other ways such as having a formulation with a different solubility agent. FIG. 2 includes blocks 200-268. Though arranged substantially serially in the example of FIG. 2, other examples may reorder the blocks, omit one or more blocks, and/or execute two or more blocks in parallel using multiple processors or a single processor organized as two or more virtual machines or sub-processors. Moreover, still other examples can implement the blocks as one or more specific interconnected hardware or integrated circuit modules with related control and data signals communicated between and through the modules. Thus, any process flow is applicable to software, firmware, hardware, and hybrid implementations.

Referring now to FIG. 2, there are three stages to a drug distribution process 200, the intake stage 220, the processing stage 240, and the dispensing stage 260. At 222, an enrollment form and/or a prescription is received into the system. In an embodiment, the system is an exclusive central pharmacy. In another embodiment, the term “exclusive” means that the pharmacy is the only pharmacy in existence that is authorized to distribute the prescription drug.

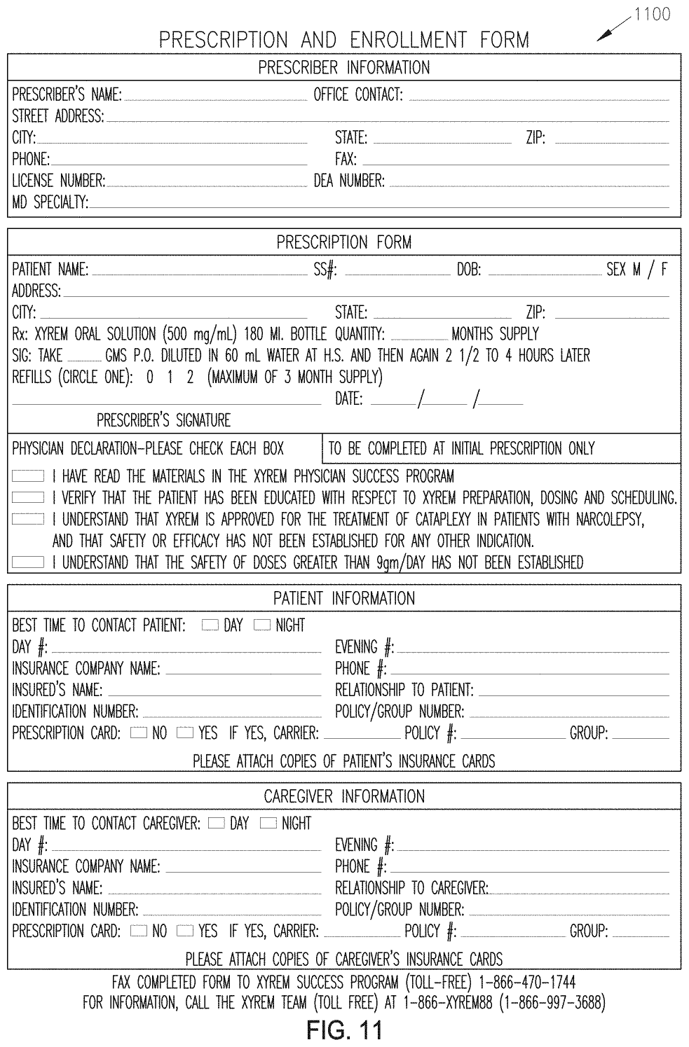

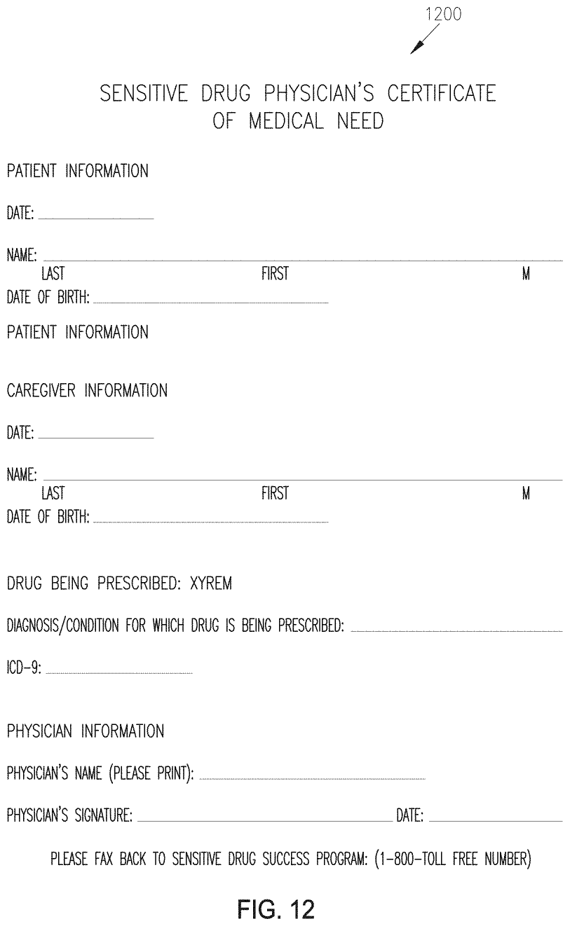

If a patient has not received a prescription through the system before, the patient must first be enrolled. Examples of enrollment forms are illustrated in FIGS. 10 and 11. If the patient has already been enrolled, and the patient is now receiving a new prescription or a prescription refill, then it is the prescription or refill request that is received at 222. In both instances, it is a physician, approved to prescribe the prescription drug, who enrolls and/or writes the prescription for the patient. The system 200 can handle and process many patients, many medical doctors who are authorized to prescribe a drug, and many different types of drugs. In an embodiment, at 222, the prescription forms used by a medical doctor to prescribe one or more of the drugs will be a different form for each different type of drug. For example, in the patient counseling checklist of FIG. 10, the form can list the particular drug and/or formulation at the top of the form such as XYREM or XYREM low sodium formulation.

In an embodiment, the medical doctor, if the medical doctor has a website signature, can fill out a prescription form via the central pharmacy website. On the website, the medical doctor can fill out the prescription form, print out the prescription form, and fax the prescription form to the central pharmacy from the website. Alternatively, the medical doctor can download the prescription form from a government regulatory site, such as the Food and Drug Administration (FDA) website (or download it from the central pharmacy website), and then mail the prescription form to the central pharmacy. In yet another embodiment, the medical doctor can simply fill in the prescription form by hand, and mail or fax the completed prescription form to the central pharmacy. The method of transmission from the medical doctor to the central pharmacy is not critical. However, the appropriate prescription form for the specific prescription drug should be used.

At 224, there is a shareholder intake (enrollment) operation, where the exclusive central pharmacy verifies that the proper prescription form and the enrollment form are on file for the stakeholders. The stakeholders are the patient, the medical doctor who writes the prescription, and the exclusive central pharmacy. In another embodiment, there is a caregiver stakeholder, who provides care and oversight to a particular patient, such as a pediatric patient. At 224, the system verifies that the prescription for the specific prescription drug that is being prescribed has been submitted by the medical doctor. As noted, there may be different prescription forms for each type of different prescription drug. Each one of these different prescription forms can be identified by a different icon, a different drug name or trademark, and/or a different look and feel. These differences will assist in making sure that the proper form is used for the specific prescription drug and the specific patient.

At 226, after the stakeholder intake of operation 224, the exclusive central pharmacy finalizes the stakeholder intake. This can be referred to as enrollment finalization, that is, the finalization of the enrolling of the patient. As noted in the previous paragraph, stakeholder intake is any one of the stakeholders (central pharmacy, medical doctor, patient) being enrolled to distribute or receive the prescription drug through the exclusive central pharmacy. The patient cannot move through the system and receive any prescription drug until enrollment is completed. At 226, enrollment is finalized once the central pharmacy has received the completed enrollment forms from the prescriber. It is noted that a patient cannot enroll himself or herself; enrollment must be done by the medical doctor. Another manner of stating this is that patients are passively enrolled by the prescribing medical doctor. Once a patient is completely enrolled and finalized with the exclusive central pharmacy, the patient can receive the prescription drug that is identified in the prescription provided by the medical doctor.

It is noted that the exclusive central pharmacy, in enrolling a patient, can experience status changes. For example, the status could be that the central pharmacy is still in the process of gathering information for enrollment for the patient because the medical doctor has not provided all the patient information. Also, the medical doctor could be properly enrolled, but the patient's enrollment may not be complete. That is, the two enrollments are not completed at the same time. In such an instance, enrollment has not been finalized at 226, and the exclusive central pharmacy will reach out to the prescribing medical doctor and request that the medical doctor provide the missing information for the patient. This incomplete enrollment could be because the medical doctor has not submitted the patient enrollment form yet, or it could be because the medical doctor has submitted a patient enrollment form, but has failed to fill in some of the patient information. Once the exclusive central pharmacy has collected all the prescribing medical doctor and patient information, the exclusive central pharmacy at operation 226 sets its system and database to indicate that enrollment is complete for the medical doctor and/or patient.

After the exclusive central pharmacy verities that enrollment is complete at 226, the exclusive central pharmacy proceeds to product specific patient counseling for the patient at 228 The product specific patient counseling is performed for any new patient, for any existing patient who has not taken the prescription drug for a particular period of time (e,g., six months), any patient who begins taking a new formulation of the prescription drug, any patient who begins taking a new prescription drug, and/or any patient who reports a new comorbid medical condition.

The product specific patient counseling at 228 is specifically tailored to the drug that is being prescribed to a patient. As noted above, in an embodiment, the exclusive central pharmacy has a patient counseling checklist, an embodiment of which is illustrated in FIG. 10, and each checklist includes distinct language that is directed to the specific prescription drug. For example, a prescription drug may contain sodium as a solubility agent, and the checklist would then include counseling regarding the amount of salt in the patient's diet. Similarly, if a patient is switching from one formulation of a drug to another formulation, there may have to be some specific counseling regarding the taking of the drug itself, and/or the food that can be ingested in connection with the new formulation. This can be referred to as a changing product check (CPC). An example of counseling verbiage that could be included with a change from one formulation (of Xyrem for example) to another formulation is outlined in the following paragraph.

Patients changing between [formulation 1] and [formulation 2]. Patients may change therapies while enrolled in the [formulation 1] and [formulation 2] REMS provided that the following requirements have been met. The patient does not have overlapping active prescriptions of [formulation 1] and [formulation 2]. If a patient is changing between [formulation 1] and [formulation 2] for the first time, or the patient has not changed between them within the past 6 months, the certified pharmacy must contact the patient or caregiver and inform the patient or caregiver the following. The safe use and administration instructions and the REMS requirements for both products are the same. [Formulation 1] and [formulation 2] may taste differently, but work the same. [Formulation 1] and [formulation 2] use the same syringe. It is important that [formulation 1] or [formulation 2] are taken as instructed by you or your child's doctor. Never take both [formulation 1] and [formulation 2] at the same time. If the patient is changing from [formulation 1] to [formulation 2], the [formulation 2] doses should be taken at least 2 hours after eating until a stable dose is achieved. Thereafter, [formulation 2] can be taken with or without food. If the patient is changing from [formulation 2] to [formulation 1], [formulation 1] doses should be taken at least 2 hours after eating. [Formulation 1] contains a high amount of sodium (salt) and may not be right for them, particularly if the patient is on a salt-restricted diet or has heart failure, hypertension, or compromised renal function. Determine if the patient is on a salt-restricted diet, has high blood pressure, heart failure, or kidney problems. If the patient has any of these conditions, the patient and/or caregiver must complete Steps 1, 3, 4, and 5 on the counseling checklist. See FIGS. 10A-10F.

The product specific patient counseling can include a warning that the patient or caregiver should never attempt to alter a dose of the prescription drug. This includes instances when a patient changes from one formulation of a prescription drug to another formulation of the prescription drug. Notwithstanding, the patient is normally counseled on the correct preparation and administration of the specific prescription drug. The patient can also be counseled that the specific prescription drug is a controlled substance, and that any misplaced or lost drug must be reported to the central pharmacy. The patient can also be counseled on side effects of the specific prescription drug. Patients should be counseled to report to the central pharmacy if the patient is pregnant or plans to become pregnant, if the patient is breastfeeding, if the patient is experiencing depression, if the patient has liver problems, if the patient experiences sleep apnea, if the patient has mental health problems, and/or if the patient experiences episodes of sleep walking. During the product specific patient counseling, the patient can also be asked if he or she is taking any other prescription drug.

A drug take-back operation is executed by the exclusive central pharmacy at 230. In an embodiment, this take-back operation is invoked when a patient is moving between prescription drugs, including different formulations of a specific prescription drug. The take-back operation assists with any excess prescription drug that a patient may have on hand. The take-back operation 230 can be particularly useful in a situation where a patient is potentially changing between prescription drugs before the patient has completed taking all the drug in his or her current prescription. The take-back operation provides a way for such a patient to properly dispose of any unused or expired prescription drugs. In an embodiment, the take-back operation 230 causes the exclusive central pharmacy to contact the patient, and instruct the patient on how to and where to send the unused or expired prescription drug. Upon receipt, the central pharmacy will destroy the unused or expired prescription drug, and make note in its exclusive computer database that the unused drug has been received from the patient and properly disposed. It is noted that the exclusive central pharmacy may or may not delay shipping the new prescription drug to the patient if the patient does not return any unused or expired prescription. This may depend on the particular circumstances at hand, including requirements and regulations of a government agency, and/or if the patient has any history of abuse, misuse, or diversion of a prescription drug as determined using the data in the exclusive computer database.

At the prescription processing operation 232, the prescription presented for a patient is validated for authentication of the prescriber, a valid date, and prescribing information including prescriber Drug Enforcement Administration (DEA) and/or National Provider Identifier (NPI) information, patient information, and dosing details. Any faxed prescriptions are confirmed by matching the fax banner to the prescriber information on file to ensure the prescription was sent from the office(s) on file for that prescriber. Upon validation of the prescription, the process requires the prescription to be uploaded to the system for processing for the specified product. Each valid prescription is filled according to the prescriber's instructions (for up to six months or until a new prescription is presented for that patient). The prescription for that patient then enters the specified product pathway 234 determined by the current prescription information provided for that patient.

The product pathway determination operation 234 is a decision operation that determines, based on which drug is being prescribed, what the next step will be. For example, as illustrated in FIG. 2, if a patient has been prescribed prescription drug A, then operation 240 will be executed. If prescribed prescription drug B, then operation 242 will be executed. If prescribed prescription drug C, then operation 244 will be executed. In each of operations 240, 242, and 244, that is the point at which the exclusive central pharmacy specifically fills the prescription for the drugs A, B, and C. At this point, the verification checks that are done across the central pharmacy are done as described elsewhere in this disclosure. While the operations in 240, 242, and 244 are not necessarily drug specific, in an embodiment, the operations can perform checks based on the prescription drug that is being processed. For example, as noted elsewhere in this disclosure, the central pharmacy will check to make sure that the prescribing medical doctor has a valid license, that is, that there is no improper activity associated with the prescribing medical doctor. As another example, the central pharmacy will check to make sure that there are no concomitant medications for the patient before the prescription drug is shipped to the patient. Also, the central pharmacy will make sure that the patient is not a duplicate patient in the exclusive database, so that there are no overlapping prescriptions sent to the same patient. The check for overlapping prescriptions can be executed in a couple of different ways. The check for overlapping prescriptions segregates the different prescription drugs, and checks not just for one formulation of a prescription drug, but it also checks for a new formulation that is on hand to make sure there are not overlaps between the former formulation and the new formulation. These checks are illustrated as operation 248 in FIG. 2. However, instead of just checking one product for overlapping prescriptions, the exclusive central pharmacy checks across multiple prescription drugs for overlapping prescriptions. The duplicate patient check is to make sure that there is not a patient who is listed more than once in the exclusive computer database, which could lead to a patient receiving two prescriptions. This assures that only one prescription is filled for any patient. That is, there is only one patient in the exclusive computer database with that information and there is only one patient then who will receive that prescription. With the personal identifying information in the exclusive computer database (e.g., social security number), there is no chance that two people have the same identifying information that would result in the same prescription being sent to two different people. In short, the central pharmacy verifies that a patient is not duplicated in the exclusive database such that the duplicate patient potentially could receive additional prescriptions. Consequently, when a patient is first enrolled in the exclusive central pharmacy, or that patient gets married and changes their name, checks are made to ensure that a duplicate record is not created in the exclusive computer database for that one patient.

After such a name change, the patient is now in the exclusive database under a different name, and it is possible that that information has not been aligned or has not been merged, perhaps because the change and merge occurred at the same time. The exclusive central pharmacy therefore determines if a prescription has recently been received, for example within the previous seven days. The central pharmacy then verifies that the patient is not in the exclusive database twice, such that the central pharmacy would accidentally send the prescription to the patient twice—once for the previous name and a second time for the new name.

Once all the checks are performed, the central pharmacy can proceed to the dispensing of the prescription drug at 262. The dispensing operations 262 include preparing the prescription drug such as checking to verify that the written prescription and the physical drug match. The central pharmacy also can separate the prescription into different size containers, such as 90 ml bottles and 180 ml bottles. At 264, the central pharmacy verifies that all the safe use conditions, which are basically the groupings of all the safety checks that have to be met in order for the prescription to be filled, have been met. Once the central pharmacy verifies that the operations are complete and correct, the central pharmacy schedules the shipment and the prescription drug is sent to the patient. At 266, the patient receives the prescription drug along with any specific materials about the use of the specific drug.

At 268, the exclusive central pharmacy provides information to the patient about the drug take-back program. Specifically, in an embodiment, information about the prescription drug take-back is included in the shipment of the prescription drug.

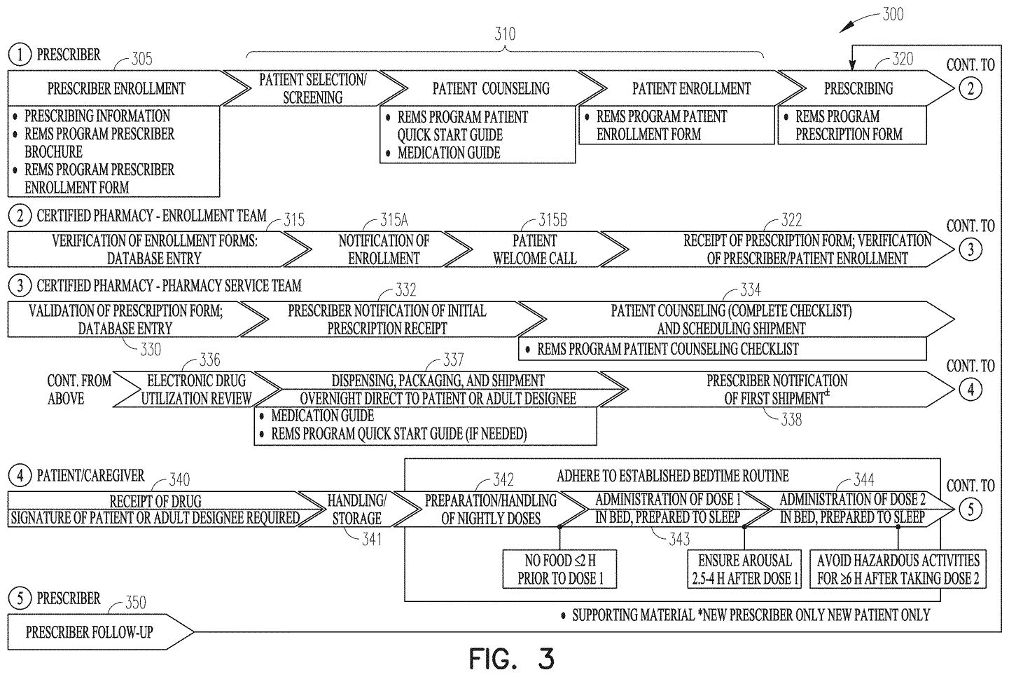

Another example embodiment of a restricted distribution program for a sensitive drug is illustrated in FIG. 3, and like with FIG. 2, can also be referred to as a Risk Evaluation and Mitigation Strategy (REMS). The REMS system 300 begins with prescriber and patient enrollment, screening, counseling, and educating. Specifically, as illustrated at 305, a prescriber is enrolled in the REMS program. The prescriber enrollment 305 includes providing prescribing information and a prescribing program brochure to the potential prescriber. After review of the information and brochure, the prescriber can fill out a prescriber enrollment form. This prescriber enrollment form is submitted to a certified, central pharmacy, as will be discussed in more detail in connection with operation 315.

In a similar fashion, as indicated at 310, potential patients, including pediatric patients and caregivers associated with those pediatric patients, are screened, selected, counseled, and enrolled. The screening, selecting, counseling, and enrolling procedures include providing to the patient a patient quick start guide, a medication guide, and a patient enrollment form. The patient quick start guide, medication guide, and brochure instruct and educate the patient on the proper use, dosing, and handling of the sensitive drug. After review of the patient quick start guide, the medication guide, and the brochure, the patient can fill out a patient enrollment form. This patient enrollment form is submitted to a certified, central pharmacy, as will be discussed in more detail in connection with operation 315.

As indicated at 315, a certified, central pharmacy includes an enrollment team. The enrollment team verifies the completion of the prescriber and patient enrollment forms, and enters the prescriber, patient, and if present caregiver, information into the exclusive database of the certified, central pharmacy. At 315A, the certified, central pharmacy notifies the prescriber or patient, as the case may be, of the successful enrollment, and as indicated at 315B, a welcome message is transmitted to the patient such as via a telephone call, an email message, a mailed letter, a text message, or any other form of suitable communication.

After successful enrollment of a prescriber, the enrolled prescriber can prescribe the sensitive drug to one or more enrolled patients. Similarly, after successful enrollment of a patient, the enrolled patient can be prescribed the sensitive drug by an enrolled prescriber. The writing of a prescription for the sensitive drug is done using a REMS program prescription form (320). At 322, the prescriber transmits the prescription form to the certified, central pharmacy. Upon receipt of the prescription form, the certified, central pharmacy verifies the prescriber and patient information on the prescription form with the prescriber and patient information in the certified, central pharmacy's exclusive computer database. As noted previously, the prescriber and patient information in the certified, central pharmacy's exclusive database was extracted from the prescriber and patient enrollment forms.

Operation 330 commences the duties of the certified, central pharmacy's services team. Specifically, at 330, the certified, central pharmacy's services team validates the non-prescriber and non-patient prescription information (that is, the actual prescription drug information) on the prescription form. After the prescription information is validated, the prescription information is entered into the certified, central pharmacy's exclusive database. As noted elsewhere in this specification, requiring the entry into the certified, central pharmacy's exclusive database of all prescribers prescribing the prescription drug and all patients being prescribed the prescription drug assists in preventing abuse, misuse, and/or diversion of the prescription drug. At 332, the certified, central pharmacy notifies the prescriber that the certified, central pharmacy has received the prescription request from the prescriber.

At 334, the certified, central pharmacy verifies that the patient has completed the required counseling, and/or provides additional counseling and responds to any questions or concerns of the patient. To ease the verification process for the certified, central pharmacy, the certified, central pharmacy can employ a patient counseling checklist, such as the checklist illustrated in FIG. 10, which can include items such as verifying that the patient has read the educational materials, has completed all necessary forms, and has completed any tests and/or assessments. That is, the checklist ensures that the patient is educated and counseled about safe use and handling of the sensitive drug and about the requirements of the REMS program, and documents information about concomitant medication use and comorbidities.

At 336, the certified, central pharmacy uses its computer systems and its exclusive database to conduct an electronic drug utilization review. In an embodiment, the electronic drug utilization review queries the exclusive database to determine what, if any, other pharmaceuticals the patient is presently taking or has taken in the past. For any pharmaceuticals that the patient is taking or has taken, the system checks its database to determine if any of these pharmaceuticals may cause an adverse reaction if taken in conjunction with the sensitive drug. If there is a potential for adverse reaction, the system notifies personnel of the certified, central pharmacy, and the personnel can take the appropriate action.

If the electronic drug utilization review does not identify any other pharmaceuticals that could cause an adverse reaction in connection with the sensitive drug, or any potential adverse reactions with other pharmaceuticals have been adequately addressed by certified, central pharmacy personnel, then the certified, central pharmacy proceeds with the dispensing, packaging, and shipment of the sensitive drug at 337. The dispensing involves placing the correct amount of the sensitive drug into the correct number and size of containers. The packaging of the sensitive drug includes adding the sensitive drug containers into a packaging/shipping box or other container, and adding incidentals such as measuring cups or other apparatus, a copy of the medication guide, and a copy of the patient quick start guide, and a copy of the caregiver brochure that is specific to pediatric and other special needs patients. The entire package is then shipped to the patient. In an embodiment, this shipment is via an overnight carrier.

At 338, the prescriber is notified of the shipment of the sensitive drug to the patient. Such notification can occur for only the first shipment of the sensitive drug, or for any shipment of the sensitive drug to the patient. Such notification enables the prescriber to follow the prescription history of the sensitive drug to the patient, and to more easily become aware of any abuse, misuse, and/or diversion of the sensitive drug.

Beginning at 340, operations are described that relate to the receipt of the sensitive drug by the patient, and the administering of the sensitive drug to the patient. Specifically, at 340, the patient receives the sensitive drug via a delivery service, and signs for the sensitive drug before the delivery service leaves the sensitive drug with the patient. The delivery service can be Priority Mail or other delivery service of the U.S. Post Office. The delivery service can also be a commercial delivery service. Two items are noteworthy here. The sensitive drug is only available through the certified, central pharmacy and its associated exclusive database; it is not available for delivery through a retail pharmacy. Also, the certified, central pharmacy can receive an immediate electronic communication from the delivery service that the sensitive drug has been delivered to and signed/accepted by the patient. The certified, central pharmacy can then also inform the prescriber that the sensitive drug has been successfully delivered to the patient. These procedures and sub-procedures contribute to the certified, central pharmacy's ability to prevent abuse, misuse, and/or diversion of the sensitive drug.

At 341, the patient appropriately stores the sensitive drug. The appropriate storage of the sensitive drug is outlined and explained in the medication guide and the patient quick start guide. Storage requirements can involve such matters as the proper environment for the sensitive drug (light, temperature, humidity, etc.)and prevention of access of the drug by other persons (for example, storing the sensitive drug in a locked cabinet).

At 342, the patient prepares a dosage of the sensitive drug. In a simple situation, this may only involve removing a pill, tablet, or capsule from its container, and taking or administering the sensitive drug to the patient with the proper type and amount of fluids and food. In a more complex situation, there may be multiple doses within a particular time period, wherein that time period may be during nighttime hours, and the sensitive drug may be in a liquid form that requires a measuring of the proper amount of the sensitive drug.

More specifically, as illustrated at operations 343 and 344, there is an administering of the sensitive drug in a first dosage and a second dosage. For example, as could be explained in the medication guide or the patient quick start guide, no food should be consumed during a certain time period prior to administering the sensitive drug. For example, no food should be consumed two hours (or less) before the sensitive drug is taken. If the sensitive drug relates to the treatment of sleep disorders, it may be required that the sensitive drug is only taken in bed, just prior to laying down in bed to sleep for the night. Additionally, if the second dose is to be taken before the patient wakes in the morning, the patient should set an alarm so that the patient can wake up and take the second dose of the sensitive drug. The second dose should be at the patient's bedside, so that the patient can take the second dose in bed, and immediately lay down again and return to sleep for the night. There may be additional provisos such as not operating any vehicles or machinery for a certain time period after any dose. The proper dosage regime of operations 343 and 344 are once again outlined and explained in the medication guide and/or the patient quick start guide.

At 350, the prescriber follows up with the patient to verify that the patient is taking the sensitive drug in the appropriate manner, and to determine if there are any other issues with the patient that need to be addressed. In addition to the prescriber follow-up, the certified, central pharmacy may also follow up with the patient to determine that the sensitive drug is being properly administered and that there are no issues with the patient and/or caregiver.

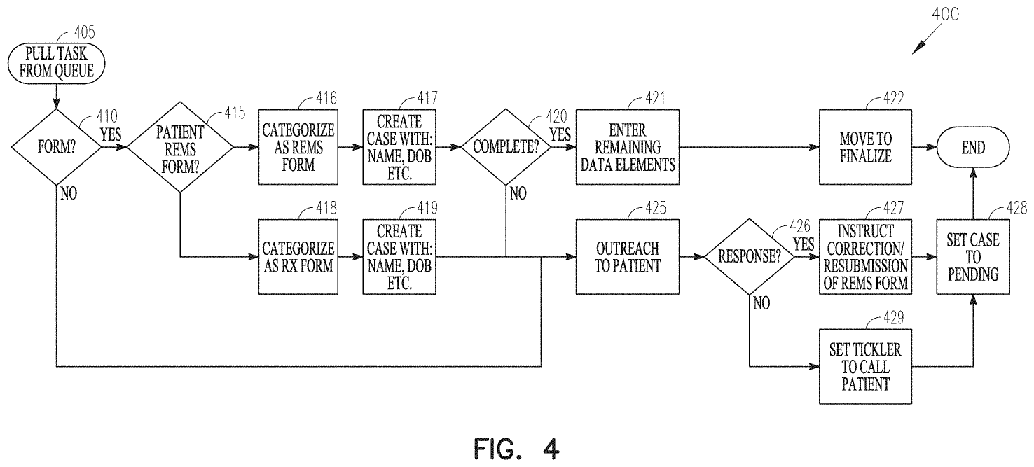

Yet another embodiment of a REMS restricted distribution program 400 for a sensitive drug is illustrated in FIG. 4. At 405, a task is pulled from a queue. This task queue represents actions that must be taken by the certified, central pharmacy in relation to distributing the sensitive drug, from enrollment to shipping to follow-up.

At decision block 410, the computer system of the certified, central pharmacy determines if the task in the queue relates to some type of form. If the task does not relate to a form, the system moves on to an outreach to the patient (and/or associated caregiver) as noted in operation 425, which is discussed in detail below. If the task does relate to a form, then at 415, the certified, central pharmacy determines if the form is a REMS form (that is, an enrollment form). If the task is a REMS form, then the form is formally categorized as a REMS form at 416, and at 417, a case is created for the patient that includes all the information needed to enroll the patient in the REMS system such as name, date of birth, gender, height, weight, etc.

If the certified, central pharmacy determines at operation 415 that the task is not a REMS form, then the certified, central pharmacy categorizes the form as a prescription form at 418. That is, the form at 418 is from a prescriber who is prescribing a sensitive drug to a patient. Then at 419, a case is created for this particular drug for this particular patient for this particular prescription.

At decision block 420, the certified, central pharmacy determines whether the creation of the REMS case at operation 417 has been completed. If the creation of the REMS case has not been completed, the remaining data are entered at operation 421. Such remaining data can include any data relating to the new patient enrollee that was not previously available, such as marital status. After entering the remaining data at operation 421, a final review of the completed REMS form is performed at 422.

At operation 425, a patient outreach is performed. This outreach occurs both after the certified, central pharmacy determines at 420 that the REMS form has not been completed, and after the certified, central pharmacy creates at 419 a prescription form that was written by a particular prescriber for a particular sensitive drug for a particular patient. For the uncompleted REMS form, the patient outreach may be to determine the information that is missing from the REMS form. For the prescription form, the outreach may be able to determine if the patient has begun taking any other prescription drugs, since the patient's enrollment in the REMS program, wherein such other prescription drugs could possibly negatively affect the patient if the patient begins taking the new sensitive drug.

At decision block 426, the certified, central pharmacy determines whether there was a. response from the patient to the outreach to the patient at 425. If there was a patient response, then at 427 the REMS form is first corrected and/or updated, and at 428, the patient's case is set to pending. That is, it is waiting for a prescription to be received at the certified, central pharmacy or it is waiting for the certified, central pharmacy to act on distributing a pending prescription. If a response was not received from the patient at 426, then at 429, the certified, central pharmacy dockets a note to follow up with a call to the patient.

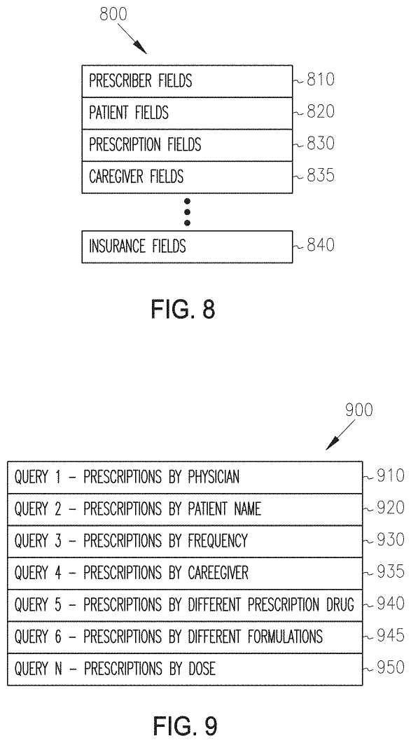

In an embodiment, the central database described above can be a relational database running on the system of FIG. 1, or a server-based system having a similar architecture coupled to workstations via a network, as represented by communications 160. The database is likely stored in storage 140, and contains multiple fields of information as indicated at 800 in FIG. 8. The organization and groupings of the fields are shown in one format for convenience. It is recognized that many different organizations or schemas may be utilized. In one embodiment, the groups of fields comprise prescriber fields 810, patient fields 820, prescription fields 830, caregiver fields 835, and insurance fields 840. For purposes of illustration, all the entries described with respect to the above processes are included in the fields. In further embodiments, no such groupings are made, and the data are organized in a different manner.

Several queries are illustrated at 900 in FIG. 9. There may be many other queries as required by individual state reporting requirements. A first query at 910 is used to identify prescriptions written by physician. The queries may be written in structured query language, natural query languages or in any other manner compatible with the database. A second query 920 is used to pull information from the database related to prescriptions by patient name. A third query 930 is used to determine prescriptions by frequency. A fourth query 935 is used to determine prescriptions by caregiver. A fifth query 940 is used to determine the prescriptions by each different prescription drug. A sixth query 945 is used to determine the prescriptions by each different formulation, and an n^{th} query finds prescriptions by dose at 950. Using query languages combined with the depth of data in the central database allows many other methods of investigating for potential abuse of the drugs, such as artificial intelligence techniques as described more fully elsewhere herein. The central database ensures that all prescriptions, prescribers, patients, and caregivers are tracked and subject to such investigations. In further embodiments, the central database may be distributed among multiple computers provided a query operates over all data relating to such prescriptions, prescribers, patients, and caregivers for the drug.

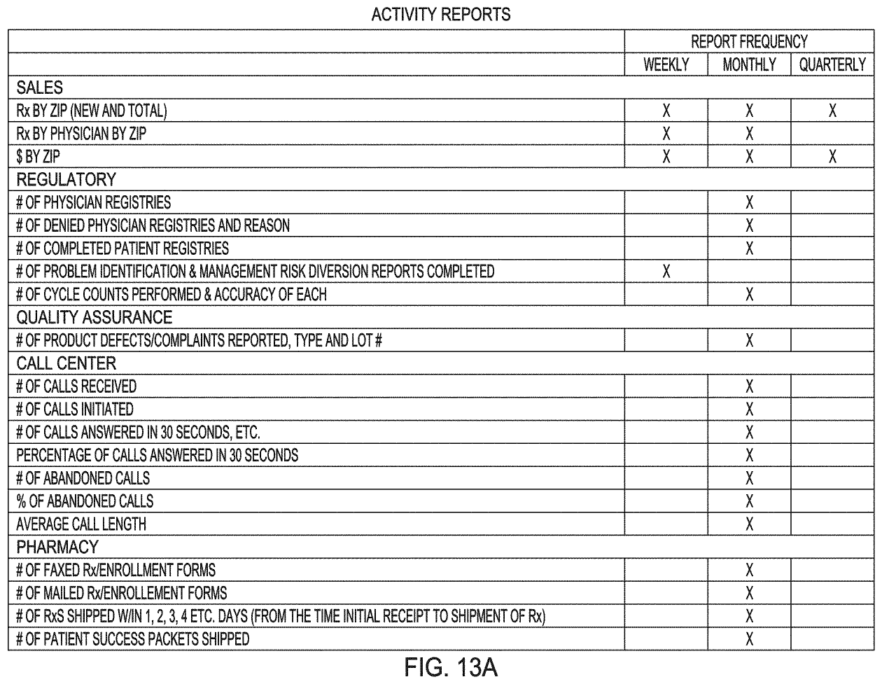

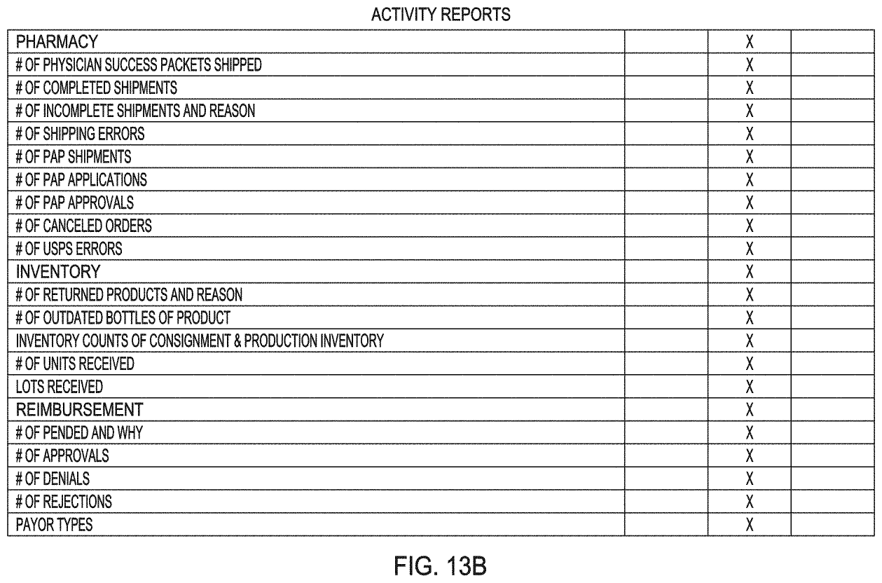

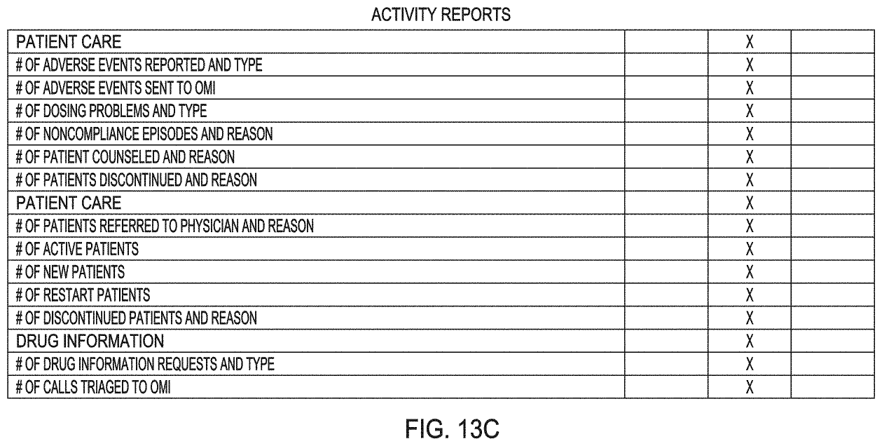

FIGS. 13A, 13B, and 13C are descriptions of sample reports obtained by querying a central database having fields represented in FIG. 8. The activities are grouped by sales, regulatory, quality assurance, call center, pharmacy, inventory, reimbursement, patient care and drug information. Each report has an associated frequency or frequencies. The reports are obtained by running queries against the database, with the queries written in one of many query languages.

While an embodiment of the invention has been described with respect to a Schedule III drug, it is useful for other sensitive drugs that are DEA or Federally scheduled drugs in Schedule II-V, as well as still other sensitive drugs where multiple controls are desired for distribution and use.

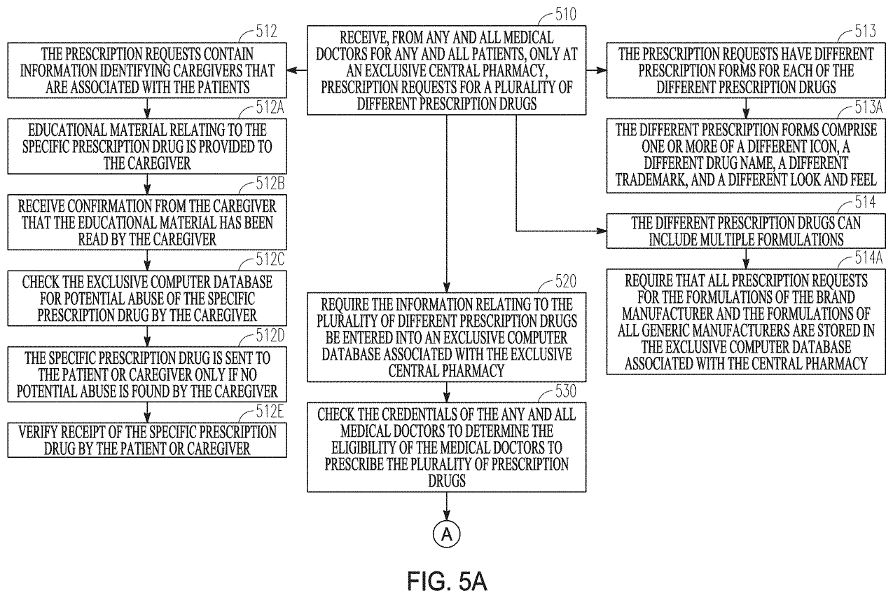

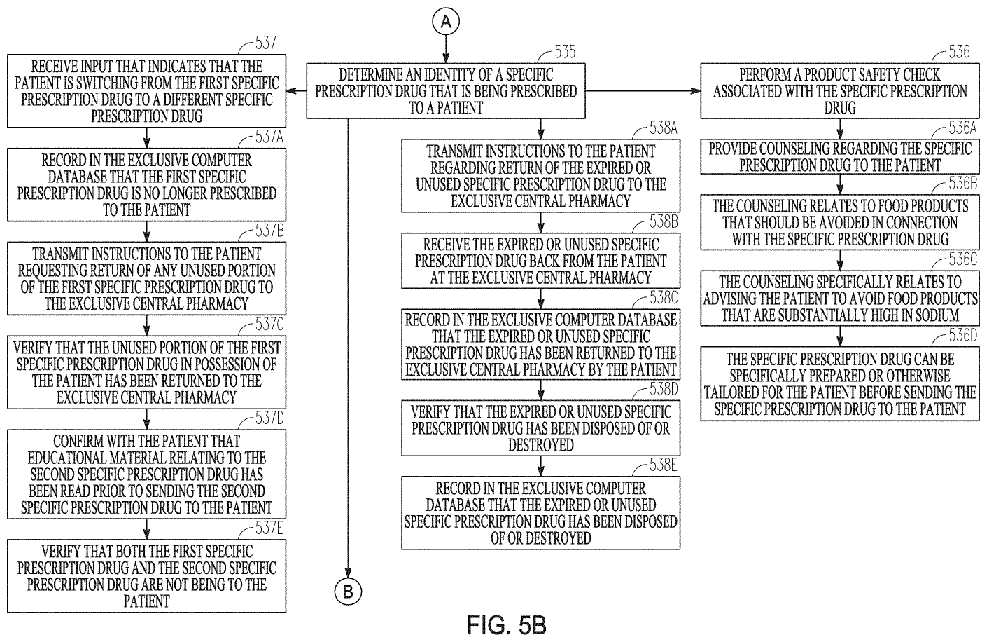

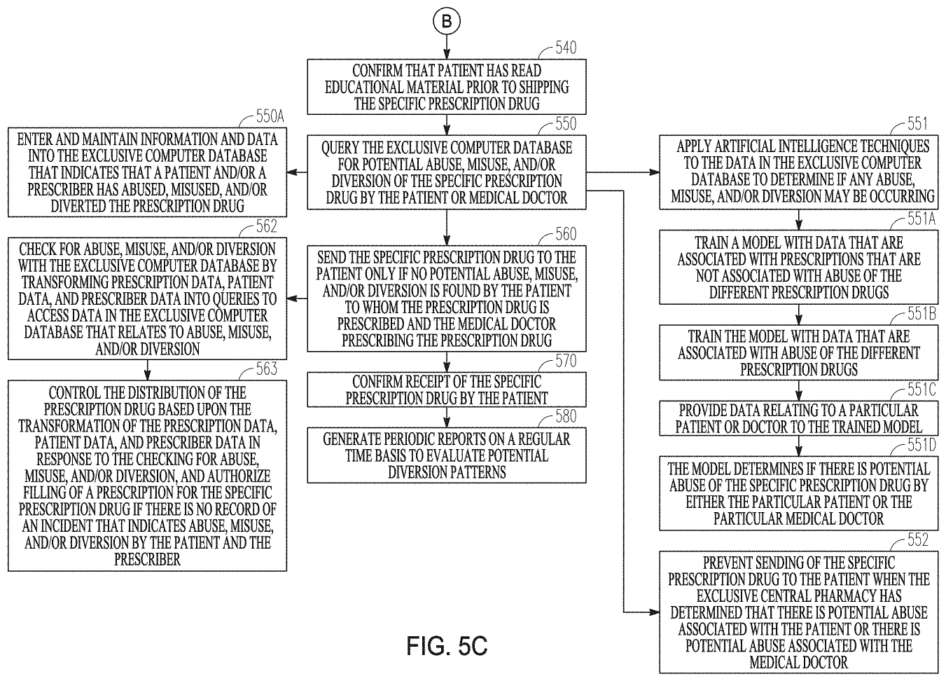

FIGS. 5A, 5B, 5C, 6A, 6B, and 7 are block diagrams illustrating features and operations of a system and method of distributing a plurality of different prescription drugs that are under exclusive control of an exclusive central pharmacy, and monitoring the patient and/or the prescriber to identify potential abuse, misuse, and/or diversion of one or more of the plurality of prescription drugs. FIGS. 5A, 5B, 5C, 6A, 6B, and 7 include process blocks 510-580, 610-60B2, and 710-784 respectively. Though arranged somewhat serially in the examples of FIGS. 5A, 5B, 5C, 6A, 6B, and 7, other examples may reorder the blocks, omit one or more blocks, and/or execute two or more blocks in parallel using multiple processors or a single processor organized as two or more virtual machines or sub-processors. Moreover, still other examples can implement the blocks as one or more specific interconnected hardware or integrated circuit modules with related control and data signals communicated between and through the modules. Thus, any process flow is applicable to software, firmware, hardware, and hybrid implementations.

Referring now specifically to FIGS. 5A, 5B, and 5C, at 510, prescription requests for a plurality of different prescription drugs are received into a computer processor. A different prescription drug can be an entirely different drug with a different indication, or a different formulation of a drug with the same indication. The prescription requests for the different prescription drugs are received from any and all medical doctors allowed to prescribe one or more of the plurality of prescription drugs. The prescription requests are received for any and all patients that are being prescribed one or more of the plurality of prescription drugs. All these prescription requests are received only at an exclusive central pharmacy (also referred to as a certified, central pharmacy). In an embodiment, an exclusive central pharmacy is a pharmacy that is the only pharmacy that is permitted to process prescriptions for the plurality of different prescription drugs. By requiring that all of the prescription requests for the plurality of different prescription drugs are received only at the exclusive central pharmacy, the system and method establish a high degree of control over the distribution of the prescription drugs, and the system and method can more easily identify potential abuse, misuse, and/or diversion of the prescription drugs, which will be explained in more detail in the following paragraphs. Credentials of the any and all medical doctors are also entered into the computer processor. The prescription requests contain information identifying patients and one or more of the plurality of different prescription drugs.

At 513, the prescription requests have different prescription forms for each of the different prescription drugs. And as indicated at 513A, the different prescription forms comprise one or more of a different icon, a different drug name, a different trademark, and a different look and feel. An example of a form with a different drug name and/or trademark is illustrated in FIG. 10.

As disclosed in operation 520, the system and method require that the information relating to the plurality of different prescription drugs is entered into an exclusive computer database that is associated with the exclusive central pharmacy. The exclusive central pharmacy can then use the exclusive computer database to analyze the prescription, patient, and medical doctor data to identify potential abuse, misuse, and/or diversion situations. The requirement that all prescriptions for the prescription drug are processed only by the exclusive central pharmacy using only the exclusive computer database makes the system's ability to identify potential abuse, misuse, and/or diversions more robust by accessing the protected data in the exclusive computer database that has been collected over a period of time and using that data for proactively monitoring patient safety.

At 530, the computer processor checks the credentials of the any and all medical doctors to determine the eligibility of the medical doctors to prescribe one or more of the plurality of prescription drugs. At 535, the computer processor determines, from the prescription request, an identity of a specific prescription drug, out of the plurality of different prescription drugs, that is being prescribed to a patient. At 536, a product safety check that is associated with the specific prescription drug is performed. In an embodiment, as illustrated in 536A, counseling regarding the specific prescription drug is provided to the patient. This counseling can use the product specific checklist that is illustrated in FIG. 10. In another embodiment, at 536B, the counseling to the patient relates to food products that the patient should avoid in connection with administering the specific prescription drug. At 536C, the counseling specifically relates to advising the patient to avoid food products that are substantially high in sodium. Foods that are substantially high in sodium can include any foods that are salty to the taste, such as salty snacks, French fries and other fast foods, and even home-made foods to which a good deal of salt is added. In connection with a consideration of any specific foods that should be ingested or avoided with the specific prescription drug, as indicated at 536D, the specific prescription drug can be specifically prepared or otherwise tailored for the patient before sending the specific prescription drug to the patient. For example, a low sodium formulation of the specific prescription drug can be prepared for the patient.

At 540, it is confirmed with a patient that educational material has been read prior to shipping the specific prescription drug to the patient. If more than one specific prescription drug is prescribed to a patient, then it is confirmed that the patient has read all educational material for all prescription drugs.

At 550, the computer processor queries the exclusive computer database for potential abuse, misuse, and/or diversion of the specific prescription drug by the patient or medical doctor. For example, the computer processor can check to see if a patient is requesting too many early refills, and/or if a medical doctor is prescribing an inordinate number of prescriptions to an inordinate number of patients. At 550A, information and data are entered into and maintained in the exclusive computer database that indicates that a patient and/or a prescriber has abused, misused, and/or diverted the prescription drug. This information can be gleaned from checking the database via queries, as just discussed in connection with operation 550. Alternatively, this information can be directly entered by an official of the central pharmacy after identifying a potential abuse, misuse, and/or diversion of the prescription drug via some means other than a. database query.

In another embodiment, as indicated at 551, one or more artificial intelligence techniques are applied to the data in the exclusive computer database to determine if any abuse, misuse, and/or diversion may be occurring. For example, at 551A, a model is first trained with data that are associated with prescriptions that are not associated with abuse of one or more of the different prescription drugs. Then, at 551B, the model is trained with data that are associated with abuse of one or more of the different prescription drugs. At 551C, data relating to a particular patient or doctor are provided to the trained model. Then, at 551D, the model determines if there is potential abuse of the specific prescription drug by either the particular patient or the particular medical doctor.

Irrespective of the particular manner in which the system determines if there has been any potential abuse, misuse, and/or diversion, the system at 552 prevents the sending of the specific prescription drug to the patient when the exclusive central pharmacy has determined that there is potential abuse associated with the patient or there is potential abuse associated with the medical doctor.

At 560, the specific prescription drug is sent to the patient via the U.S. Mail, or otherwise delivered to the patient, only if no potential abuse, misuse, and/or diversion is found by patient to whom the prescription drug is prescribed and the medical doctor prescribing the prescription drug. As noted throughout this specification, potential abuse, misuse, and/or diversion by the patient could be identified by a patient requesting early refills or claiming that prescriptions have been stolen, lost, or spilled. Potential abuse, misuse, and/or diversion by the medical doctor could be identified, for example, if the medical doctor writes prescriptions for early refills for several patients.

In another embodiment, as outlined at 562, the system and method check for abuse, misuse, and/or diversion with the exclusive computer database by transforming prescription data, patient data, and prescriber data into one or more queries to access data in the exclusive computer database that relate to abuse, misuse, and/or diversion. The results of the queries can indicate whether abuse, misuse, and/or diversion has potentially occurred and should be investigated. In another embodiment, as indicated at 563, the system and method control the distribution of the prescription drug based upon the transformation of the prescription data, patient data, and prescriber data in response to the checking for abuse, misuse, and/or diversion, and authorize filling of a prescription for the specific prescription drug if there is no record of an incident that indicates abuse, misuse, and/or diversion by the patient and the prescriber. If there is a record of such incident, the exclusive computer database indicates that such incident has been investigated, and the exclusive computer database indicates that such incident does not involve abuse, misuse, and/or diversion.

At 570, the system and method confirm receipt of the specific prescription drug by the patient. Such confirmation can be accomplished by having the patient electronically sign using a mobile device of the delivery service, and receiving at the computer processor of the exclusive central pharmacy confirmation directly from the mobile device of the delivery service (or an intermediate server of the delivery service or other party).

At 580, the system and method generate with the computer processor and the exclusive computer database periodic reports on a regular time basis to evaluate potential diversion patterns. A regular time basis has equally spaced time intervals between reports, and such reports are not generated in response to a specific report or incident of abuse, misuse, and/or diversion. A potential diversion pattern could be identifying that a patient has claimed that there have been three spillage incidents of the prescription drug within the last three months. Those of skill in the art will realize that other patterns could be identified. At 581, the system and method selectively block shipment of the prescription drug to the patient based on a determination of potential abuse, misuse, and/or diversion by the patient.

At 537, the computer processor receives input that indicates that the patient is switching from the first specific prescription drug to a different specific prescription drug. As previously noted, this new different prescription drug can be a different prescription drug with a different active ingredient and a different indication, or a prescription drug with a different formulation and the same active ingredient and indication. At 537A, the computer processor records in the exclusive computer database that the first specific prescription drug is no longer prescribed to the patient. At 537B, the computer processor transmits instructions to the patient requesting return of any unused portion of the first specific prescription drug to the exclusive central pharmacy. At 537C, the computer processor verifies that the unused portion of the first specific prescription drug in possession of the patient has been returned to the exclusive central pharmacy. Then, at 537D, after verifying that the unused portion of the first specific prescription drug in the possession of the patient has been returned to the exclusive central pharmacy, the computer processor confirms with the patient that educational material relating to the second specific prescription drug has been read prior to sending the second specific prescription drug to the patient. In an embodiment, at 537E, the computer processor verifies that the exclusive central pharmacy is not concurrently sending both the first specific prescription drug and the second specific prescription drug to the patient.

In another embodiment similar to the embodiment outlined in operations 537A-537E, the system handles the situation wherein a prescription drug has reached its expiration date, or the situation wherein the prescription drug remaining in the possession of the patient will not be used by the patient. In this embodiment, at 538A, the system transmits instructions to the patient regarding return of the expired or unused specific prescription drug to the exclusive central pharmacy. At 538B, the expired or unused specific prescription drug is received back from the patient at the exclusive central pharmacy. At 538C, the system records in the exclusive computer database that the expired or unused specific prescription drug has been returned to the exclusive central pharmacy by the patient. At 538D, it is verified that the expired or unused specific prescription drug has been disposed of or destroyed, and at 538E, it is recorded in the exclusive computer database that the expired or unused specific prescription drug has been disposed of or destroyed.

As indicated at 512, in an embodiment, the prescription requests contain information identifying caregivers that are associated with the patients. In such situations, as further indicated at 512A, educational material relating to the specific prescription drug is provided to the caregiver. At 512B, the system, prior to sending the specific drug to the patient, receives confirmation from the caregiver that the educational material has been read by the caregiver. Thereafter, at 512C, the system checks the exclusive computer database for potential abuse of the specific prescription drug by the caregiver. At 512D, the specific prescription drug is sent to the patient or caregiver only if no potential abuse is found by the caregiver. At 512E, the system verifies or confirms receipt of the specific prescription drug by the patient or caregiver.

As noted at 514, one or more of the different prescription drugs can include multiple formulations. In an embodiment, these different formulations include the same active ingredient, and have the same indications. In another embodiment, these different formulations can be from a brand manufacturer and a generic manufacturer. In such a situation, the system and method can determine that the potential abuse is associated with the formulation from the brand manufacturer or the formulation from the generic manufacturer. The ability to make this distinction can assist in identifying whether a particular formulation is susceptible to abuse, misuse, and/or diversion. The ability to make this determination and/or distinction is made possible by the system and method requiring that all the prescription requests for the formulations of the brand manufacturer and the formulations of all generic manufacturers are stored in the exclusive computer database associated with the central pharmacy (514A).

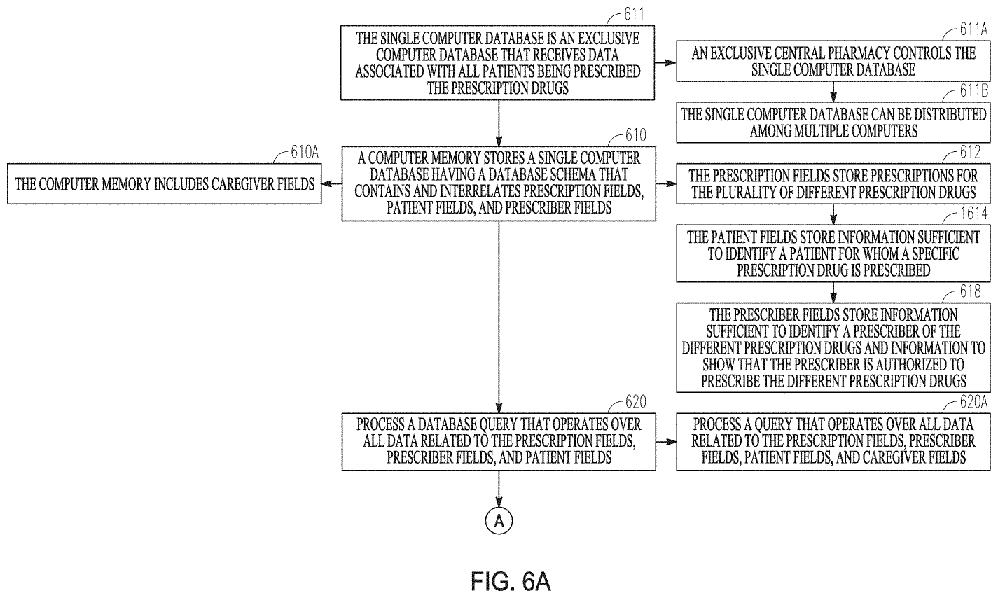

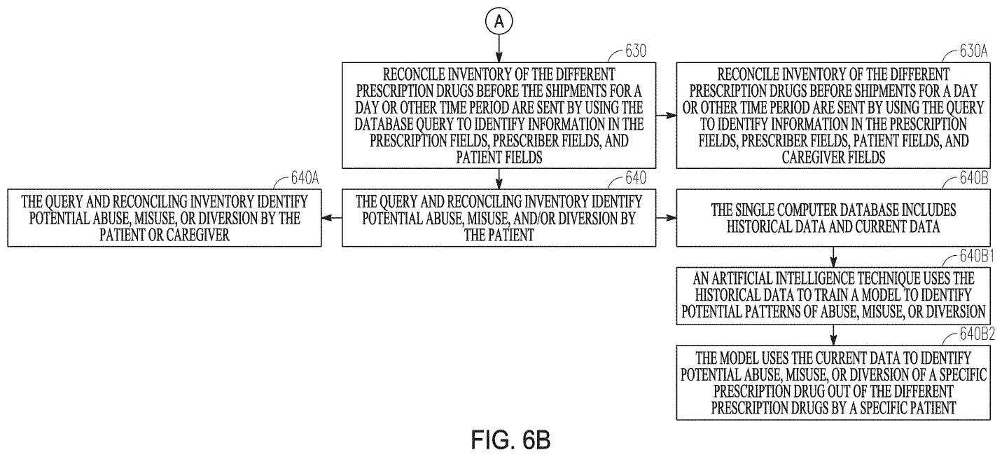

FIGS. 6A and 6B are a block diagram illustrating other embodiments of a system and method for distribution of a plurality of different prescription drugs that have a potential for abuse, misuse, and/or diversion. The embodiments of FIGS. 6A and 6B include one or more computer memories for storing a single computer database having a database schema that contains and interrelates prescription fields, patient fields, and prescriber fields (610). The prescription fields are contained within the database schema, and as indicated at 612, the prescription fields store prescriptions for the plurality of different prescription drugs with the potential for abuse, misuse and/or diversion. The patient fields are contained within the database schema, and as indicated at 614, the patient fields store information sufficient to identify a patient for whom a specific prescription drug out of the plurality of different prescription drugs is prescribed. The prescriber fields are contained within the database schema, and as indicated at 618, the prescriber fields store information sufficient to identify a physician or other prescriber of the plurality of different prescription drugs and information to show that the physician or other prescriber is authorized to prescribe the plurality of different prescription drugs.

The embodiments of FIGS. 6A and 6B, at 620, process a database query that operates over all data related to the prescription fields, prescriber fields, and patient fields, and at 630, the system and method reconcile inventory of the plurality of different prescription drugs before the shipments for a day or other time period are sent by using the database query to identify information in the prescription fields, prescriber fields, and patient fields. The database query and the reconciling inventory identify potential abuse, misuse, and/or diversion by the patient (640). The system and method then, based on that identification of the potential abuse, misuse, and/or diversion, notify the physician, who is interrelated with the patient through the schema of the single computer database, about the potential abuse, misuse, and/or diversion. The physician can then take action to address the potential abuse, misuse, and/or diversion by contacting the patient, or take other action to investigate the identified potential abuse, misuse, and/or diversion. In an embodiment, the inventory reconciliation is performed where current inventory is counted and reconciled with database quantities before shipments for a day or other time period are sent, and the system and method selectively block shipment of the specific prescription drug to the patient based on the inventory reconciliation.

In another embodiment, at 610A, the one or more computer memories include caregiver fields. The caregiver fields, which are contained within the database schema, store information sufficient to identify a caregiver that is interrelated with the patient through the schema of the single computer database. At 620A, the data processor processes a query that operates over all data related to the prescription fields (for the plurality of different prescription drugs), prescriber fields, patient fields, and caregiver fields. At 630A, the data processor reconciles inventory of the plurality of different prescription drugs before shipments for a day or other time period are sent by using said database query to identify information in the prescription fields, prescriber fields, patient fields, and caregiver fields. At 640A, the database query and the reconciling of inventory identify potential abuse, misuse, or diversion by the patient or caregiver. Thereafter, if potential abuse, misuse, and/or diversion is found, the data processor can be used to notify the physician that is interrelated with the patient through the schema of the single computer database.

As indicated at 611, the single computer database is an exclusive computer database that receives data associated with all patients being prescribed one or more of the plurality of prescription drugs. And as indicated at 611A, an exclusive central pharmacy controls the single computer database. As indicated at 611B, the single computer database can be distributed among multiple computers. In such an embodiment, the database query that checks for potential abuse, misuse, and/or diversion of the plurality of different prescriptions drug operates over all data relating to the prescription fields, prescriber fields, and patient fields.

In another embodiment, as indicated at 640B, the single computer database includes historical data and current data. At 640B1, an artificial intelligence technique uses the historical data to train a model to identify potential patterns of abuse, misuse, or diversion, and at 640B2, the model uses the current data to identify potential abuse, misuse, or diversion of a specific prescription drug out of the plurality of different prescription drugs by a specific patient.

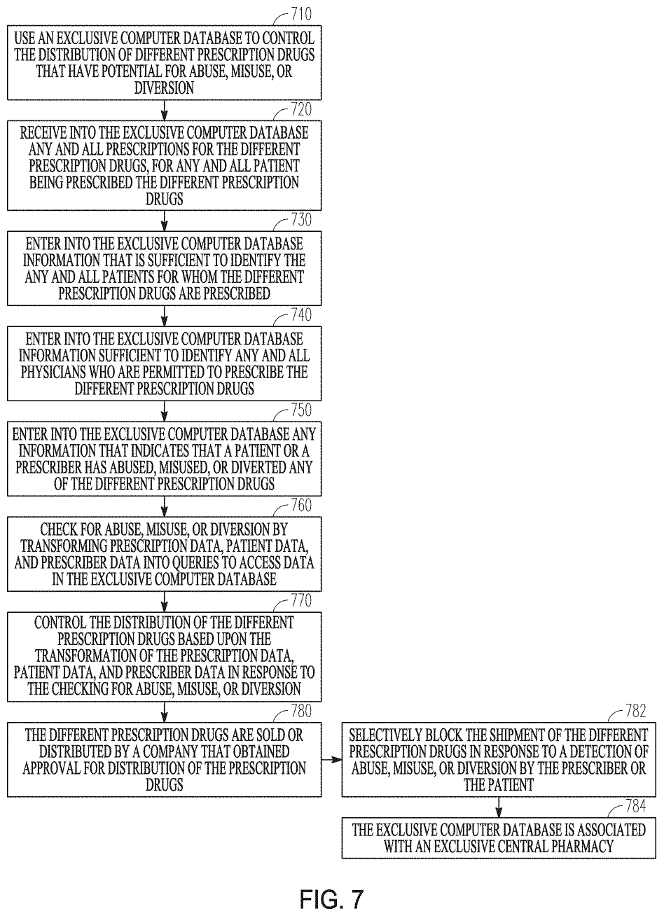

FIG. 7 is a block diagram illustrating other embodiments of a system and method for distribution of a plurality of different prescription drugs that have a potential for abuse, misuse, and/or diversion. The embodiments of FIG. 7 includes a computer processor that uses an exclusive computer database to control the distribution of a plurality of different prescription drugs that have potential for abuse, misuse, or diversion (710). At 720, any and all prescriptions for the plurality of different prescription drugs with the potential for abuse, misuse, or diversion, for any and all patient being prescribed one or more of the plurality of different prescription drugs, are received into the computer processor and the exclusive computer database. At 730, information that is sufficient to identify the any and all patients for whom the plurality of different prescription drugs is prescribed is entered into the computer processor and the exclusive computer database. At 740, information sufficient to identify any and all physicians or other prescribers of the plurality of different prescription drugs and information to show that the any and all physicians or other prescribers are authorized to prescribe the plurality of different prescription drugs are entered into the computer processor and the exclusive computer database. At 750, any information that indicates that a patient or a prescriber has abused, misused, or diverted any of the plurality of different prescription drugs is entered into and maintained using the computer processor and the exclusive computer database. At 760, the computer processor checks for abuse, misuse, or diversion with the exclusive computer database by transforming prescription data, patient data, and prescriber data into one or more queries to access data in the exclusive computer database that relates to abuse, misuse, or diversion. The queries indicate whether investigatable abuse, misuse, or diversion has occurred. At 770, the computer processor controls the distribution of the plurality of different prescription drugs based upon the transformation of the prescription data, patient data, and prescriber data in response to the checking for abuse, misuse, or diversion. The computer processor authorizes the filling of a prescription for one or more of the plurality of different prescription drugs if there is no record of an incident that indicates abuse, misuse, or diversion by the patient or the prescriber. If there is a record of such incident, the exclusive computer database indicates that such incident has been investigated, and the exclusive computer database indicates that such incident does not involve abuse, misuse or diversion.

At 780, the plurality of different prescription drugs is sold or distributed by a company that obtained approval for distribution of the plurality of different prescription drugs. The exclusive computer database of the company that obtained approval for distribution of the plurality of different prescription drugs is the only database in existence for the company's plurality of different prescription drugs. Because of this system architecture, all prescriptions for the company's plurality of different prescription drugs are processed only by the exclusive computer database of the company.

At 782, the shipment of the one or more plurality of different prescription drugs is selectively blocked in response to a detection of abuse, misuse, or diversion by the prescriber or the patient. At 784, in an embodiment, the exclusive computer database is associated with an exclusive central pharmacy. In this embodiment, the inventory of the plurality of different prescription drugs is owned by a company, the inventory is consigned to the exclusive central pharmacy and managed by the exclusive central pharmacy.

As can be inferred and seen from the foregoing, a system to control the distribution of a prescription drug, which can be referred to as a Risk Evaluation and Mitigation Strategy (REMS) Program, has a goal of mitigating the risks of serious adverse outcomes resulting from inappropriate prescribing, misuse, abuse, and/or diversion of the prescription drug. This goal is achieved by (1) informing prescribers, pharmacists, patients, and caregivers of the risks, contraindications, potential for abuse, misuse, diversion, and/or overdose, and the safe use, handling, and storage of the prescription drug, and (2) ensuring that pharmacy controls exist prior to filling prescriptions that screen for concomitant use of potentially interacting agents, monitoring for inappropriate prescribing, and monitoring and notifying prescribers about concomitant use of contraindicated medications or signs of potential abuse, misuse, and/or diversion.

It should be understood that there exist implementations of other variations and modifications of the invention and its various aspects, as may be readily apparent, for example, to those of ordinary skill in the art, and that the invention is not limited by specific embodiments described herein. Features and embodiments described above may be combined with each other in different combinations. It is therefore contemplated to cover any and all modifications, variations, combinations or equivalents that fall within the scope of the present invention.

The Abstract is provided to comply with 37 C.F.R. § 1.72(b) and will allow the reader to quickly ascertain the nature and gist of the technical disclosure. It is submitted with the understanding that it will not be used to interpret or limit the scope or meaning of the claims.

In the foregoing description of the embodiments, various features are grouped together in a single embodiment for the purpose of streamlining the disclosure. This method of disclosure is not to be interpreted as reflecting that the claimed embodiments have more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate example embodiment.

1. A computerized process comprising distributing a plurality of different prescription drugs under exclusive control of an exclusive central pharmacy, the method comprising:
receiving in a computer processor all prescription requests, from any and all medical doctors allowed to prescribe the plurality of different prescription drugs, for any and all patients being prescribed the plurality of different prescription drugs, only at the exclusive central pharmacy, the prescription requests containing information identifying patients, the plurality of different prescription drugs, and various credentials of the any and all medical doctors;
requiring entering of the information into an exclusive computer database associated with the exclusive central pharmacy for analysis of potential abuse situations, such that all prescriptions for the plurality of different prescriptions drugs are processed only by the exclusive central pharmacy using only the exclusive computer database;
using the computer processor to check the credentials of the any and all medical doctors to determine the eligibility of the medical doctors to prescribe the plurality of different prescription drugs;
determining an identity of a specific prescription drug, out of the plurality of different prescription drugs, that is being prescribed to a patient;
verifying that educational material relating to the specific prescription drug has been provided to the patient;
prior to sending the specific prescription drug to the patient, receiving confirmation from the patient that the educational material relating to the specific prescription drug has been read by the patient;
checking the exclusive computer database for potential abuse of the specific prescription drug by the patient or a medical doctor prescribing the specific prescription drug;
sending the specific prescription drug to the patient only if no potential abuse is found by both the patient and the medical doctor;
confirming receipt of the specific prescription drug by the patient; and
generating with the computer processor using the exclusive computer database periodic reports on a regular time basis to evaluate potential diversion patterns.

2. The computerized process of claim 1, comprising:
receiving input indicating that the patient is switching from the specific prescription drug to a second specific prescription drug;
recording in the exclusive computer database that the specific prescription drug is no longer prescribed to the patient;
transmitting instructions to the patient regarding return of any unused portion of the specific prescription drug to the exclusive central pharmacy;
verifying that the unused portion of the specific prescription drug in possession of the patient has been returned to the exclusive central pharmacy; and
after verifying that the unused portion of the specific prescription drug in the possession of the patient has been returned to the exclusive central pharmacy, confirming with the patient that educational material relating to the second specific prescription drug has been read prior to sending the second specific prescription drug to the patient.

3. The computerized process of claim 2, comprising verifying that the exclusive central pharmacy is not concurrently sending both the specific prescription drug and the second specific prescription drug to the patient.

4. The computerized process of claim 1, comprising executing a product safety check associated with the specific prescription drug.

5. The computerized process of claim 1, comprising preparing the specific prescription drug in a manner that is tailored to the specific prescription drug before sending the specific prescription drug to the patient.

6. The computerized process of claim 1, comprising:
receiving an indication that the specific prescription drug in the possession of the patient has expired or will not be used by the patient;
transmitting instructions to the patient regarding return of the expired or unused specific prescription drug to the exclusive central pharmacy;
receiving at the exclusive central pharmacy the expired or unused specific prescription drug from the patient;
recording in the exclusive computer database that the expired or unused specific prescription drug has been returned to the exclusive central pharmacy by the patient;
verifying that the expired or unused specific prescription drug has been disposed of or destroyed; and
recording in the exclusive computer database that the expired or unused specific prescription drug has been disposed of or destroyed.

7. The computerized process of claim 1, comprising preventing the sending of the specific prescription drug to the patient when the exclusive central pharmacy has determined that there is potential abuse associated with the patient or there is potential abuse associated with the medical doctor.

8. The computerized process of claim 1, comprising providing counseling to the patient relating to the specific prescription drug.

9. The computerized process of claim 8, wherein the counseling to the patient relates to food products that the patient should avoid in connection with administering the specific prescription drug.

10. The computerized process of claim 9, wherein the food products comprise substantially high levels of sodium.

11. The computerized process of claim 1, wherein the prescription requests contain information identifying caregivers that are associated with the patients.

12. The computerized process of claim 11, comprising providing the educational material to a caregiver associated with the patient.

13. The computerized process of claim 12, comprising receiving confirmation from the caregiver that the educational material has been read by the caregiver prior to sending the specific prescription drug to the patient or caregiver.

14. The computerized process of claim 13, comprising checking the exclusive computer database for potential abuse of the specific prescription drug by the caregiver;
sending the specific prescription drug to the patient or caregiver only if no potential abuse is found by the caregiver; and
confirming receipt of the specific prescription drug by the patient or caregiver.

15. The computerized process of claim 1, wherein the sending of the specific prescription drug to the patient comprises a use of the U.S. Mail.

16. The computerized process of claim 1, comprising using artificial intelligence to identify potential abuse of the specific prescription drug.

17. The computerized process of claim 16, wherein the artificial intelligence comprises:
training a model with data associated with prescriptions that are not associated with abuse of the plurality of different prescription drugs;
training the model with data associated with prescriptions that are associated with abuse of the plurality of different prescription drugs;
providing data to the model relating to the patient and the medical doctor; and
using the model to determine if there is potential abuse of the specific prescription drug by either the patient or the medical doctor.

18. The computerized process of claim 1, wherein the specific prescription drug comprises multiple formulations from a brand manufacturer and a generic manufacturer.

19. The computerized process of claim 18, comprising determining that the potential abuse is associated with the formulation from the brand manufacturer or the formulation from the generic manufacturer.

20. The computerized process of claim 18, wherein all the prescription requests for the multiple formulations from the brand manufacturer and the generic manufacturer are required to be stored in the exclusive central computer database.

21. The computerized process of claim 1, wherein the prescription requests comprise a different prescription form for each of the different prescription drugs.

22. The computerized process of claim 21, wherein the different prescription forms comprise one or more of a different icon, a different drug name, a different trademark, and a different look and feel.

23. The computerized process of claim 1, wherein the plurality of different prescription drugs comprises a same active ingredient.

24. A computer-implemented system for distribution of a plurality of different prescription drugs that have a potential for abuse, misuse, or diversion, comprising:
one or more computer memories for storing a single computer database having a database schema that contains and interrelates prescription fields, patient fields, and prescriber fields;
said prescription fields, contained within the database schema, storing prescriptions for the plurality of different prescription drugs with the potential for abuse, misuse, or diversion;
said patient fields, contained within the database schema, storing information sufficient to identify a patient for whom a specific prescription drug out of the plurality of different prescription drugs is prescribed;
said prescriber fields, contained within the database schema, storing information sufficient to identify a physician or other prescriber of the plurality of different prescription drugs and information to show that the physician or other prescriber is authorized to prescribe the plurality of different prescription drugs;
a data processor configured to:
process a database query that operates over all data related to the prescription fields, prescriber fields, and patient fields; and
reconcile inventory of the plurality of different prescription drugs before shipments for a day or other time period are sent by using said database query to identify information in the prescription fields, prescriber fields, and patient fields;
said database query and reconciling inventory identifying potential abuse, misuse, or diversion by the patient and being used to notify the physician that is interrelated with the patient through the schema of the single computer database.

25. The computer-implemented system of claim 24, wherein the one or more computer memories comprise caregiver fields, said caregiver fields, contained within the database schema, storing information sufficient to identify a caregiver that is interrelated with the patient through the schema of the single computer database.

26. The computer-implemented system of claim 25, wherein the data processor is configured to:
process a database query that operates over all data related to the prescription fields, prescriber fields, patient fields, and caregiver fields; and
reconcile inventory of the plurality of different prescription drugs before shipments for a day or other time period are sent by using said database query to identify information in the prescription fields, prescriber fields, patient fields, and caregiver fields;
said database query and reconciling inventory identifying potential abuse, misuse, or diversion by the patient or caregiver and being used to notify the physician that is interrelated with the patient through the schema of the single computer database.

27. The computer-implemented system of claim 24, wherein the single computer database is an exclusive computer database that receives data associated with all patients being prescribed one or more of the plurality of different prescription drugs.

28. The computer-implemented system of claim 24, wherein an exclusive central pharmacy controls the single computer database.

29. The computer-implemented system of claim 24, wherein the single computer database is distributed among multiple computers and the database query operates over all data in the multiple computers relating to said prescription fields, prescriber fields, and patient fields.

30. The computer-implemented system of claim 24, wherein the single computer database comprises historical data and current data, and wherein an artificial intelligence technique uses the historical data to train a model to identify potential patterns of abuse, misuse, or diversion; and wherein the model uses the current data to identify potential abuse, misuse, or diversion of a specific prescription drug by a specific patient.

31. A method, using a computer processor and an exclusive computer database, to control the distribution of a plurality of different prescription drugs that have potential for abuse, misuse, or diversion, comprising:
receiving into the computer processor and the exclusive computer database, for any and all patients being prescribed the plurality of different prescription drugs, any and all prescriptions for the plurality of different prescription drugs with the potential for abuse, misuse, or diversion;
entering into the computer processor and the exclusive computer database information sufficient to identify the any and all patients for whom the plurality of different prescription drugs is prescribed;
entering into the computer processor and the exclusive computer database information sufficient to identify any and all physicians or other prescribers of the plurality of different prescription drugs and information to show that the any and all physicians or other prescribers are authorized to prescribe the plurality of different prescription drugs; and
entering into and maintaining with the computer processor and the exclusive computer database any information that indicates that a patient or a prescriber has abused, misused, or diverted the plurality of different prescription drugs;
wherein the computer processor checks for abuse, misuse, or diversion with the exclusive computer database by transforming prescription data, patient data, and prescriber data into one or more queries to access data in the exclusive computer database that relates to abuse, misuse, or diversion, the queries indicating whether investigatable abuse, misuse, or diversion has occurred; and
wherein the computer processor controls the distribution of the plurality of different prescription drugs based upon the transformation of the prescription data, patient data, and prescriber data in response to the checking for abuse, misuse, or diversion, and authorizes filling of a prescription for one or more of the plurality of different prescription drugs if there is no record of an incident that indicates abuse, misuse, or diversion by the patient or the prescriber, and if there is a record of such incident, the exclusive computer database indicates that such incident has been investigated, and the exclusive computer database indicates that such incident does not involve abuse, misuse or diversion.

32. The method of claim 31, wherein the plurality of different prescription drugs is sold or distributed by a company that obtained approval for distribution of the plurality of different prescription drugs; and wherein the exclusive computer database of the company that obtained approval for distribution of the plurality of different prescription drugs is the only database in existence for the company's plurality of different prescription drugs, such that all prescriptions for the company's plurality of different prescription drugs are processed only by the exclusive computer database of the company.

33. The method of claim 31, comprising selectively blocking shipment of the one or more plurality of different prescription drugs to the patient in response to a detection of abuse, misuse, or diversion by the prescriber or the patient.

34. The method of claim 31, wherein the exclusive computer database is associated with an exclusive central pharmacy, wherein inventory of the plurality of different prescription drugs is owned by a company, and wherein the inventory is consigned to the exclusive central pharmacy and managed by the exclusive central pharmacy.
